(19) United States
(12) Patent Application Publication
(10) Pub. No.: US 2023/0243822 A1
(43) Pub. Date: Aug. 3, 2023

(54) MOLECULAR DISEASE PROFILE AND USE THEREOF FOR MONITORING AND TREATING RHEUMATAOID ARTHRITIS

(71) Applicant: JANSSEN BIOTECH, INC., Horsham, PA (US)

(72) Inventors: Matthew J. LOZA, Spring House, PA (US); Keying MA, Spring House, PA (US)

(21) Appl. No.: 18/003,185

(22) PCT Filed: Jun. 15, 2021
(86) PCT No.: PCT/IB2021/055282

§ 371 Date: Dec. 23, 2022

Related U.S. Application Data
(60) Provisional application No. 63/044,457, filed on Jun. 26, 2020.

(51) Int. Cl. G01N 33/564; G16B 40/00

(52) U.S. Cl. CPC G01N 33/564; G16B 40/00; G01N 2800/102; G01N 2800/52

(57) ABSTRACT

Biomarkers that are indicative of efficacy of a treatment for rheumatoid arthritis or for the responsiveness to a treatment regimen in a subject being treated for rheumatoid arthritis are described. Also described are probes capable of detecting the biomarkers and related methods and kits for assessing, monitoring, and selecting treatment for rheumatoid arthritis.

### CROSS-REFERENCE TO RELATED APPLICATIONS

This application claims priority to U.S. Provisional Patent Application No. 63/044,457, filed Jun. 26, 2020, the disclosure of which is incorporated herein by reference in its entirety.

### FIELD OF THE INVENTION

The present application relates generally to the fields of bioinformatics. In particular, the present teachings relate to methods and compositions for assessing, monitoring, and selecting treatment for rheumatoid arthritis (RA).

### BACKGROUND OF THE INVENTION

Rheumatoid arthritis is one of the most common systemic autoimmune diseases worldwide. It is a chronic, systemic autoimmune disorder where a patient's own immune system targets his/her own joints as well as other organs including the lung, blood vessels and pericardium, leading to inflammation of the joints (arthritis), widespread endothelial inflammation, and even destruction of joint tissue. Erosions and joint space narrowing are largely irreversible and result in cumulative disability.

Conventional treatments for RA include controlling disease activity, such as inflammation, in order to slow or prevent disease progression, in terms of tissue destruction, cartilage loss and joint erosion. However, the disease activity and disease progression can be uncoupled. The precise etiology of RA has not been fully established. While inflammation and immune dysregulation are involved in RA, the precise mechanisms and pathogenesis of RA are complex, which can be different in individual patients and may change in those subjects over time.

Considerable investment in clinical studies has been required to establish confidence that a novel treatment can be clinically efficacious for RA, e.g., by having enough patients to statistically power comparisons vs. placebo treatment and conducting the clinical study for a sufficient duration to allow clinical response to develop. A method that could shorten the duration and the number of subjects required for a clinical study while maintaining confidence in the call of likely clinical efficacy could dramatically reduce the risk and investment to next decision point for a novel experimental treatment. Preferably, such a method would be generally independent of the mechanism of action of the therapeutic class of the treatment, such that one would not need to evaluate the mechanism of action of a novel treatment in order to maintain confidence in the readout.

Such a method and related compositions are described in this application.

### SUMMARY OF THE INVENTION

In a general aspect, the application relates to an isolated set of probes for detecting a panel of biomarkers consisting of two, three, four, five, six, seven, eight, nine, ten, eleven, twelve, thirteen or fourteen biomarkers selected from: C-reactive protein pentraxin-related (CRP), serum amyloid A-1 protein (SAA), C-X-C motif chemokine 10 (CXCL10), C-X-C Motif Chemokine Ligand 13 (CXCL13), interleukin 6 (IL-6), phosphohexose isomerase (PHI), annexin I, BPI (bactericidal/permeability-increasing protein), LEAP-1 (liver-expressed antimicrobial protein), MMP-1 (metalloproteinase-1), MMP-3 (metalloproteinase-3), PBEF (pre-B-cell colony-enhancing factor 1), SP-D (surfactant protein D), and TIMP-3 (tissue inhibitor of metalloproteinase 3).

In an embodiment, the panel of biomarkers consists of CRP, SAA, CXCL10 and at least one of CXCL13, IL-6 and PHI. In another embodiment, the panel of biomarkers consists of CRP, SAA, CXCL10 and CXCL13; CRP, SAA, CXCL10 and IL-6; or CRP, SAA, CXCL10 and PHI.

In another embodiment, the probes are selected from the group consisting of an aptamer, an antibody, an affibody, a peptide and a nucleic acid. In some embodiments, the probes are labeled with one or more detectable markers.

In another aspect, the application relates to a kit comprising an isolated set of probes of the application.

In another aspect, the application relates to a method comprising:

(a) applying an isolated set of probes of the application to a biological sample to thereby measure quantitative data for each biomarker of the panel of biomarkers in the biological sample, wherein the biological sample is obtained from a subject in need of a treatment of rheumatoid arthritis at a time point T1 after the subject is treated with a therapy;

(b) obtaining a treatment dataset comprising the quantitative data for all biomarkers of the panel of biomarkers measured in (a);

(c) obtaining a baseline dataset comprising quantitative data for all biomarkers of the panel of biomarkers, preferably the baseline dataset comprises quantitative data for all biomarkers of the panel of biomarkers measured from a biological sample obtained from the subject before the subject is treated with the therapy;

(d) comparing the quantitative data in the treatment dataset with the corresponding quantitative data in the baseline dataset to obtain a change in each biomarker of the panel of biomarkers at the time point T1 after the subject is treated with the therapy; and

(e) determining the molecular disease profile (M-DP) score of the subject at the time point T1 as the median value of the changes in all biomarkers of the panel of biomarkers measured in (d).

In an embodiment, the M-DP score is determined as the median value of the log₂ transform of the ratio of the quantitative data for each biomarker in the treatment dataset over the corresponding quantitative data in the baseline dataset.

In some embodiments, the method further comprises:

(f) determining an M-DP score for the subject at at least one additional time point T2 after the subject is treated with the therapy;

(g) determining a composite M-DP score for the subject as the mean value of the M-DP scores at the time point T1 and the at least one additional time point T2 after the subject is treated with the therapy; and

(h) predicting the efficacy of the therapy in treating rheumatoid arthritis in the subject based on the composite M-DP score for the subject.

In some embodiments, the method further comprises:

(f) determining an M-DP score for each subject of a group of subjects in need of a treatment of rheumatoid arthritis at the time point T1 after the group of subjects are treated with the therapy;

(g) determining a composite M-DP score for the group of subjects at the time point T1 as the mean value of the M-DP scores for all subjects in the group of subjects at the time point T1; and

(h) predicting the efficacy of the therapy in treating rheumatoid arthritis based on the composite M-DP score.

In an embodiment, the group of subjects consists of 5 to 25 subjects, preferably 10 to 15 subjects, such as 10, 11, 12, 13, 14 or 15 subjects.

In another embodiment, the panel of biomarkers consists of CRP, SAA, CXCL10 and CXCL13.

In another embodiment, the biological sample is a serum sample.

In another embodiment, the composite M-DP score is correlated with a clinical assessment; preferably, the clinical assessment is selected from the group consisting of a DAS, a DAS28, a DAS28-CRP, a Sharp score, a tender joint count (TJC), a swollen joint count (SJC), a Clinical Disease Activity Index (CDAI), and a Simple Disease Activity Index (SDAI); more preferably the clinical assessment is the DAS28-CRP.

In another embodiment, the time point T1 is about 4 to 12 weeks after the subject is treated with the therapy; preferably the time point T1 is about 4 to 8 weeks, such as 4, 5, 6, 7, or 8 weeks, or anytime in between, after the subject is treated with the therapy.

In another embodiment, the efficacy of the therapy in treating rheumatoid arthritis is predicted based on the composite M-DP score before the efficacy is detected by a clinical assessment.

In another embodiment, the method further comprises treating the subject(s) with the therapy, if the therapy is predicted to be effective.

In another embodiment, the method further comprises treating the subject(s) with another therapy, if the therapy is predicted to be ineffective.

In another aspect, the application relates to a method of treating rheumatoid arthritis in a group of subjects in need thereof comprising:

(a) obtaining a baseline biological sample from each subject in the group of subjects;

(b) applying an isolated set of probes of the application to each of the baseline biological samples to detect a baseline expression level for each biomarker detected by the isolated set of probes;

(c) treating the subjects with a therapy for rheumatoid arthritis;

(d) obtaining a biological sample from each subject in the group of subjects at a time point T1;

(e) applying the isolated set of probes from (b) to each of the biological samples to detect an expression level for each biomarker at time point T1;

(f) determining a molecular disease profile (M-DP) score for each subject, wherein the M-DP score is the median value of the log₂ transform of the ratio of the expression level for each biomarker at time point T1 over the corresponding baseline expression level;

(g) determining a composite M-DP score for the group of subjects as the mean value of the M-DP scores for the group of subjects;

(h) continuing to treat the subjects with the therapy in (c) if the composite M-DP score is greater than one standard deviation below zero; or treating the subjects with a different therapy if the composite M-DP score is less than one standard deviation below 0, unchanged, or above zero.

In an embodiment, the time point T1 is about 4 to about 12 weeks after the baseline time; preferably wherein time point T1 is about 4 to about 8 weeks, such as 4 weeks, 5 weeks, 6 weeks, 7 weeks, 8 weeks, or any time point in between, after the baseline time.

In another embodiment, the biological sample is a serum sample.

In another embodiment, the group of subject consists of 5 to 25 subjects, preferably 10 to 15 subjects, such as 10, 11, 12, 13, 14 or 15 subjects.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A-FIG. 1E illustrate pharmacodynamic changes in M-DP scores based on a panel of 14 biomarkers (14-analyte panel), expressed as log 2(week 4/baseline) (FIG. 1A-FIG. 1D) or log 2(6-month/baseline) (FIG. 1E), values (y-axis) are reported for individual patients, stratified by treatment group (x-axis) from various clinical studies: FIG. 1A: SIRROUND-T study; FIG. 1B: GO-FURTHER study; FIG. 1C: 40346527ARA2001 study, FIG. 1D: CNT01275ARA2001 study; and FIG. 1E: TACERA study.

FIG. 2 illustrates pharmacodynamic M-DP scores across studies and treatments. M-DP scores for 14-analyte panel (left panel) and 4-analyte panel (right panel), expressed as mean±standard deviation of log 2(visit level/baseline level) values for week 4 (red) and weeks 12-26 (blue; week 24 except for GO-FURTHER at week 14, TACERA at week 26, and 40346527ARA2001 at week 12). *, p<0.05; †, p<0.0001 vs. 0-change.

FIG. 3 illustrates clinical response associations with pharmacodynamic M-DP scores for 14-analyte panel. M-DP scores for 14-analyte panel, stratified with European League Against Rheumatism (EULAR) Disease Activity Score 28-joint count (DAS28) calculated using C reactive protein (CRP) (EULAR DAS28-CRP) response (circle, good; square, moderate; triangle, no response) at week 24 (except for TACERA at week 26 and 40346527ARA2001 at week 12), expressed as mean±standard deviation of log 2(visit level/baseline level) values for week 4 (left panel) and weeks 12-26 (right panel; week 24 except for GO-FURTHER at week 14, TACERA at week 26, and 40346527ARA2001 at week 12). *, p<0.05 for Good vs. No response.

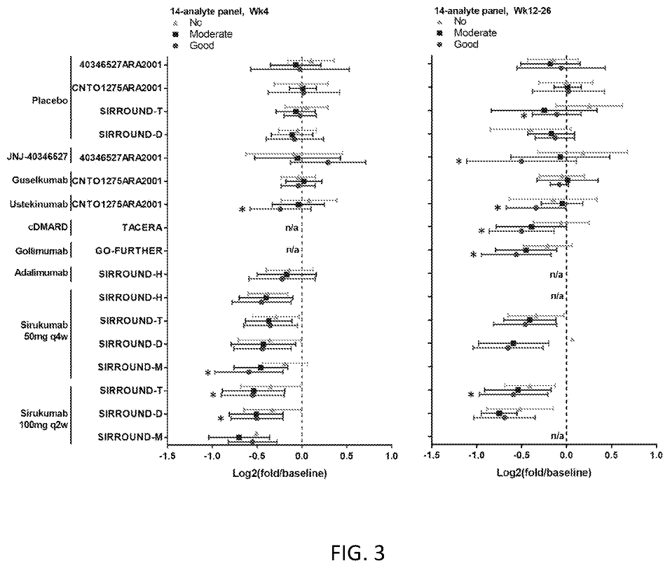

FIG. 4 illustrates clinical response associations with pharmacodynamic M-DP scores for 4-analyte panel. M-DP scores for 4-analyte panel (CRP, CXCL10, CXCL13, SAA) stratified with EULAR DAS28-CRP response (circle, good; square, moderate; triangle, no response) at week 24 (except for TACERA at week 26 and 40346527ARA2001 at week 12) expressed as mean±standard deviation of log 2(visit level/baseline level) values for week 4 (left panel) and weeks 12-26 (right panel; week 24 except for GO-FURTHER at week 14, TACERA at week 26, and 40346527ARA2001 at week 12). *, p<0.05 for Good vs. No response.

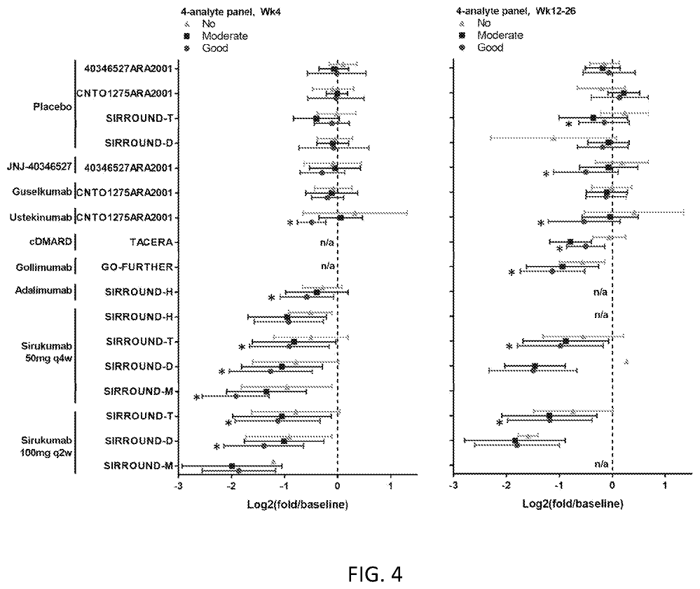

FIG. 5 illustrates the difference between good and no clinical response for pharmacodynamic M-DP scores. Differences between EULAR DAS28-CRP good vs. no response in M-DP scores at week 24 (except for TACERA at week 26 and 40346527ARA2001 at week 12) for 14-analyte panel (left panel) and 4-analyte panel (right panel) are expressed as mean difference ±standard deviation of log 2(visit level/baseline level) values for week 4 (circle) and weeks 12-26 (square; week 24 except for GO-FURTHER at week 14, TACERA at week 26, and 40346527ARA2001 at week 12). *, p<0.05 for Good vs. No response.

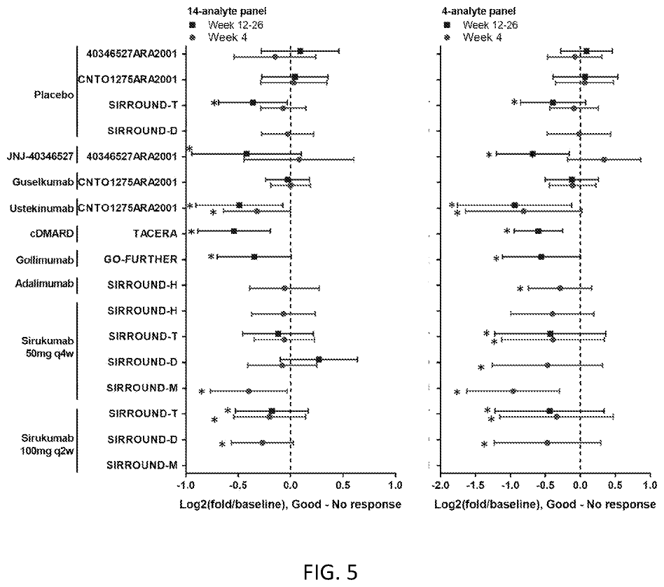

FIG. 6A-FIG. 6D illustrate the clinical response associations with pharmacodynamic M-DP scores for 4-analyte panel. M-DP scores for 4-analyte panel (CRP, CXCL10, CXCL13, SAA) stratified by treatment group and EULAR DAS28-CRP response (x-axis) at week 24 are expressed as mean±standard deviation of log 2(visit level/baseline level) values (y-axis) at week 4 for SIRROUND-T study (FIG. 6A) and CNT01275ARA2001 study (FIG. 6B), at week 14 for CO-FURTHER study (FIG. 6C), and at week 26 for RA-MAP TACERA study (FIG. 6D). *, p<0.05 for Good vs. No response.

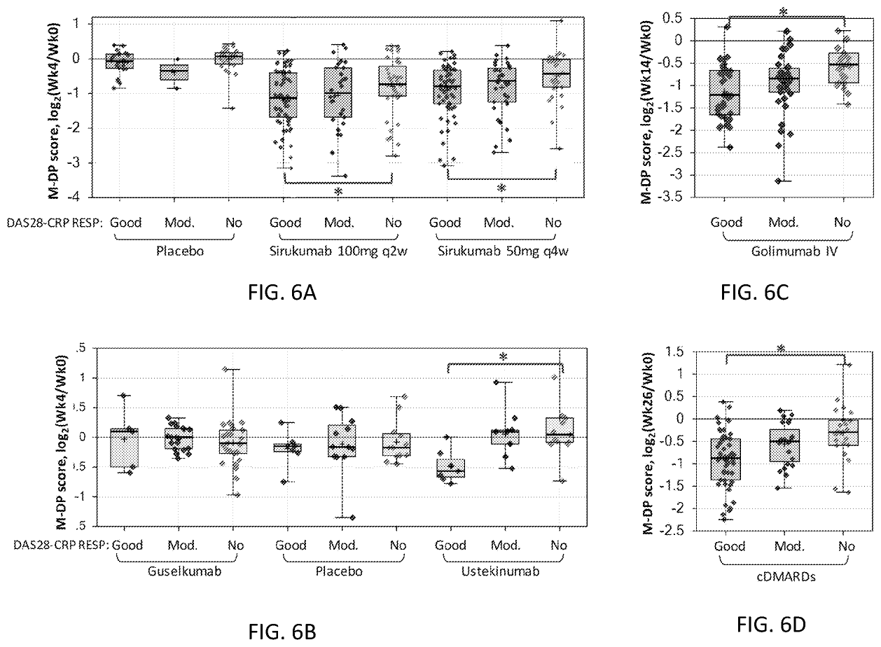

FIG. 7A-FIG. 7B illustrate the clinical response associations with pharmacodynamic M-DP scores for abatacept and rituximab treatment. M-DP scores at 3—and 6—month visits for 4-analyte panel (CRP, CXCL10, CXCL13, SAA; measured by MSD U-PLEX platform), stratified by treatment group, visit for M-DP score (month), and EULAR DAS28-CRP response at week 24 (x-axis, from bottom to top labels), are expressed as mean±standard deviation of log 2(fold/baseline) values (y-axis) at indicated visit for (FIG. 7A) abatacept and (FIG. 7B) rituximab treatment from the CORRONA CERTAIN biorepository. *, p<0.05 for comparison to 0-change (one-sample test);†, p<0.05 for good vs. no response groups.

FIG. 8 illustrates the intercorrelation of pharmacodynamic M-DP scores among analytes in the 14-analyte panel. Spearman's coefficient of correlation of ranks (RSp) between the indicated pairs of analytes in the 14-analyte M-DP panel for log 2(week 4/baseline) values from SIRROUND-T study are reported in a hierarchically-clustered heatmap.

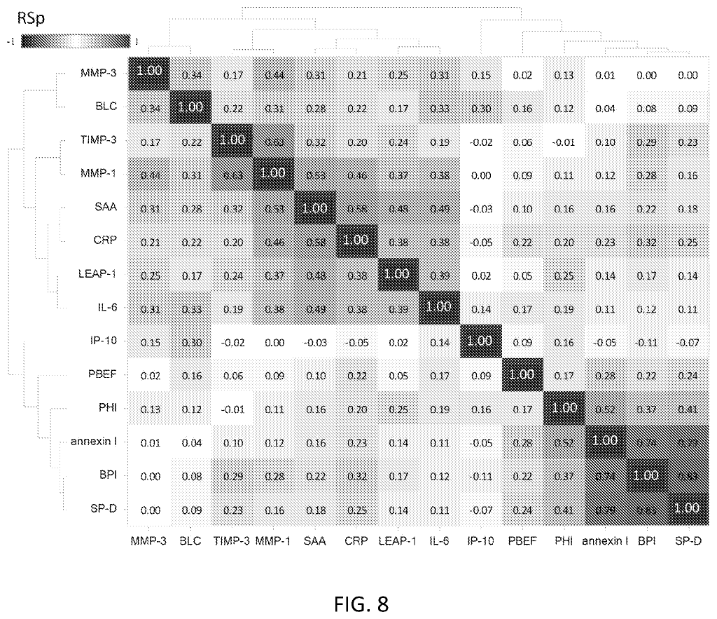

FIG. 9A-FIG. 9B illustrate the correlation between change in DAS28-CRP and pharmacodynamic M-DP scores. For SIRROUND-T study, 4-analyte M-DP scores at week 4 (FIG. 9A) and week 24 (FIG. 9B), expressed as log 2(visit level/baseline level) (y-axis) vs. week 24 changes in DAS28-CRP scores (x-axis) are shown for individual patients in the placebo (left), sirukumab 100 mg q2w (middle), and sirukumab 50 mg q4w (right) treatment groups, with symbols colored by EULAR DAS28-CRP response at week 24 (black-filled diamond, good; gray-filled triangle, moderate; white-filled diamond, no response). Pearson's correlation coefficient (p-value) for the plotted data are reported in top left of each plot.

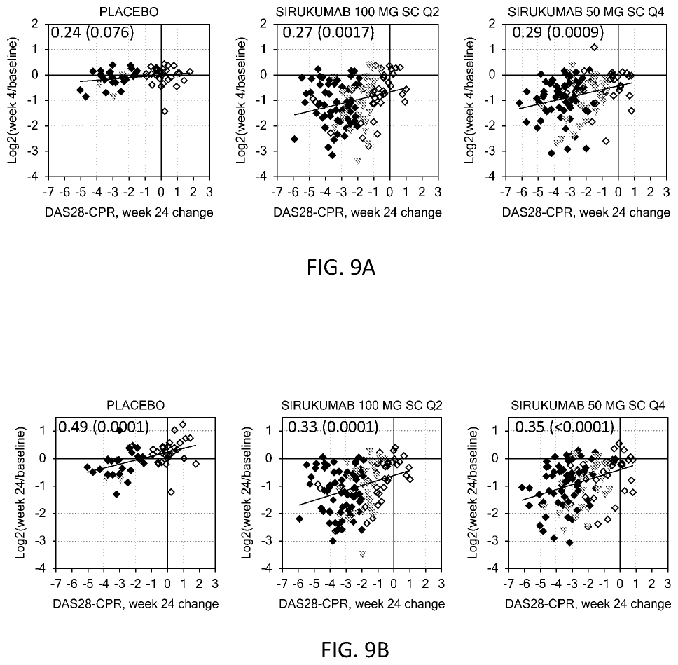

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “administering” with respect to the methods of the invention, means a method for therapeutically or prophylactically preventing, treating, or ameliorating a syndrome, disorder or disease (e.g., RA) as described herein. Such methods include administering an effective amount of said therapeutic agent at different times during the course of a therapy or concurrently in a combination form. The methods of the invention are to be understood as embracing all known therapeutic treatment regimens.

The term “antibody” herein is used in the broadest sense and specifically includes full-length monoclonal antibodies, polyclonal antibodies, and, unless otherwise stated or contradicted by context, antigen-binding fragments, antibody variants, and multispecific molecules thereof, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N. Y., (1988).

As used herein, “affibody” refers to a class of affinity proteins generated by combinatorial protein engineering to bind to a specific target protein with high affinity. Affibodies imitate monoclonal antibodies and are therefore considered antibody mimetics. Unlike antibodies, affibody molecules are small in size, around 6 kDa as compared to 150 kDa for monoclonal antibodies, and are composed of alpha helices and lack disulfide bridges. Their small size, robust behavior, and physical resiliency make them attractive for a variety of medical applications, including intracellular applications and alternative administration routes.

An “aptamer” as used herein is a short segment of DNA, RNA, or peptide that binds to a specific molecular target, such as a protein.

As used herein, “biomarker” or “analyte” refers to a gene or protein whose level of expression or concentration in a sample is altered compared to that of a normal or healthy sample or is indicative of a condition. The biomarkers disclosed herein are genes and/or proteins whose expression level or concentration or timing of expression or concentration correlates with the prognosis of rheumatoid arthritis (RA).

A “clinical assessment,” or “clinical datapoint” or “clinical endpoint,” in the context of the present teachings can refer to a measure of disease activity or severity. A clinical assessment can include a score, a value, or a set of values that can be obtained from evaluation of a sample (or population of samples) from a subject or subjects under determined conditions. A clinical assessment can also be a questionnaire completed by a subject. A clinical assessment can also be predicted by biomarkers and/or other parameters. One of skill in the art will recognize that the clinical assessment for RA, as an example, can comprise, without limitation, one or more of the following: DAS, DAS28, DAS28-CRP, Sharp score, TJC, swollen joint count (SJC), Clinical Disease Activity Index (CDAI), and Simple Disease Activity Index (SDAI).

“DAS” refers to the Disease Activity Score, a measure of the activity of RA in a subject, well-known to those of skill in the art (See D. van der Heijde et al., Ann. Rheum. Dis. 1990, 49(11):916-920). “DAS” as used herein refers to this particular Disease Activity Score. The “DAS28” involves the evaluation of 28 specific joints. It is a current standard well-recognized in research and clinical practice. Because the DAS28 is a well-recognized standard, it is often simply referred to as “DAS.” Unless otherwise specified, “DAS” herein will encompass the DAS28. A DAS28 can be calculated for an RA subject according to the standard as outlined at the das-score.nl website, maintained by the Department of Rheumatology of the University Medical Centre in Nijmegen, the Netherlands. The number of swollen joints, or swollen joint count (SJC) out of a total of 28 (SJC28), and tender joints, or tender joint count (TJC) out of a total of 28 (TJC28) in each subject is assessed. In some DAS28 calculations the subject's general health (GH) is also a factor, and can be measured on a 100 mm Visual Analogue Scale (VAS), a psychometric response scale that measures pain. GH may also be referred to herein as PG or PGA, for “patient global health assessment” (or merely “patient global assessment”). A “patient global health assessment VAS,” then, is GH measured on a Visual Analogue Scale.

“DAS28-CRP” (or “DAS28CRP”) is a DAS28 assessment calculated using C-reactive protein, pentraxin-related (CRP). CRP is produced in the liver. Normally there is little or no CRP circulating in an individual's blood serum—CRP is generally present in the body during episodes of acute inflammation or infection, so that a high or increasing amount of CRP in blood serum can be associated with acute infection or inflammation. A blood serum level of CRP greater than 1 mg/dL is usually considered high. Most inflammation and infections result in CRP levels greater than 10 mg/dL. The amount of CRP in subject sera can be quantified using, for example, the DSL-10-42100 ACTIVE® US C-Reactive Protein Enzyme-Linked Immunosorbent Assay (ELISA), developed by Diagnostics Systems Laboratories, Inc. (Webster, TX). CRP production is associated with radiological progression in RA (See M. Van Leeuwen et ah, Br. J. Rheum. 1993, 32(suppl.):9-13). CRP is thus considered an appropriate measure of RA disease activity (See R. Mallya et al., J. Rheum. 1982, 9(2):224-228, and F. Wolfe, J. Rheum. 1997, 24: 1477-1485).

The terms “Sharp score” and “modified Sharp score” each refer to radiographic scoring of individual joints. A commonly used Sharp method considers 17 areas for joint erosion and 18 areas for joint space narrowing (JSN). Each erosion scores one point, with a maximum of five points for each area (reflecting loss of more than 50% of either articular bone). Erosion scores range from 0 to 170. One point is scored for focal joint narrowing, two points for diffuse narrowing of less than 50% of the original space, and three points if the reduction is more than half of the original joint space. Ankylosis (abnormal stiffening and immobility of a joint due to fusion of the bones) is scored as four. (Sub)luxation (partial or full dislocation of a bone from a joint) is not scored. The score for JSN ranges from 0 to 144 (See Boini S and Guillemin F. Ann Rheum Dis. 2001; 60: 817-827).

“SDAI” refers to the Simplified Disease Activity Index, which combines single measures into an overall continuous measure of rheumatoid arthritis (RA) disease activity. The SDAI is calculated by adding the following items together: 28—swollen joint count (SJC28), 28— tender joint count (TJC28), patient global assessment of disease activity (PtGA or PGA) on a 10-cm visual analog scale (VAS), provider global assessment of disease activity (PrGA) on a 10-cm VAS, and C-reactive protein (CRP) level in mg/dl. The range of the SDAI is 0 to 86, with the upper limit of CRP level often defined as 10 mg/dl. See Smolen JS, et al. Rheumatology (Oxford) 2003; 42: 244-57. The “CDAI” refers to the Clinical Disease Activity Index, which is analogous to the SDAI; however, the CDAI excludes laboratory measurement of CRP level. The CDAI is calculated by adding the following items together: SJC28+TJC28+PrGA+PGA, with a range from 0 to 76. See Aletaha D, et al. Arth. Rheum. 2005, 52(9): 2625-2636.

The terms “effective amount” and “therapeutically effective amount” each mean the amount of active compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., RA). Efficacy can be measured using any of the clinical assessments described herein.

As used herein, the term “molecular disease profile score” or “M-DP score” is a score derived from a set of biomarkers or analytes dysregulated in a disease population compared to healthy controls that represents the molecular burden of the disease. Biomarkers or analytes dysregulated in a disease can be identified by analyzing and comparing biological samples collected from patients suffering from the disease with that of healthy controls. For example, an M-DP score can be derived from two or more biomarkers within a set of 14 biomarkers that were found upregulated in a phase 3 study on Sirukumab (a human anti—interleukin-6 (IL-6) monoclonal antibody) for treating RA. In some embodiments, an M-DP score is determined as the median value of the log₂ transform of the ratio of the quantitative data for each biomarker in the treatment dataset over the corresponding quantitative data in a baseline dataset.

A “population” is any grouping of subjects of like specified characteristics. The grouping could be according to, for example, clinical parameters, clinical assessments, therapeutic regimen, disease status (e.g. with disease or healthy), level of disease activity, etc. In the context of using the M-DP score in comparing therapeutic efficacy between populations, an aggregate or composite value can be determined based on the observed M-DP scores of the subjects of a population; e.g., at particular timepoints in a longitudinal study. The aggregate value can be based on, e.g., any mathematical or statistical formula useful and known in the art for arriving at a meaningful aggregate value from a collection of individual datapoints; e.g., mean, median, median of the mean, etc.

As used herein, “probe” refers to any molecule or agent that is capable of selectively binding to an intended target biomolecule. The target molecule can be a biomarker, for example, a nucleotide transcript or a protein encoded by or corresponding to a biomarker. Probes can be synthesized by one of skill in the art, or derived from appropriate biological preparations, in view of the present disclosure. Probes can be specifically designed to be labeled. Examples of molecules that can be utilized as probes include, but are not limited to, RNA, DNA, proteins, peptides, antibodies, aptamers, affibodies, and organic molecules.

A “quantitative dataset,” as used in the present teachings, refers to the data derived from, e.g., detection and composite measurements of a plurality of biomarkers (i.e., the panel of biomarkers disclosed herein) in a subject sample. The quantitative dataset can be used in the identification, monitoring and treatment of disease states, and in characterizing the biological condition of a subject.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” or “biological sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

In one general aspect, the present disclosure relates to the detection or monitoring of disease states, preferably a molecular disease profile (M-DP) of RA, in a subject, and provides methods, reagents, and kits useful for this purpose. Provided herein are probes useful for measuring quantitative data for biomarkers that are indicative and/or predictive of the M-DP of RA. In certain embodiments, the present disclosure provides an M-DP score in a subject at a specific time point that indicates the subject has developed or is at risk of developing symptoms of RA. The M-DP score can additionally be used for other purposes, such as to determine efficacy of a treatment regimen or indicate the responsiveness to the treatment for RA.

It was surprisingly discovered by the inventors of the present application that a composite M-DP score based on a 14-analyte M-DP panel for a population of RA patients is significantly decreased, e.g., as early as 4 weeks, by an effective treatment, achieving efficacy for the primary clinical endpoint at week 12-24 of treatment with various drug products (sirukumab in 4 independent studies, adalimumab, golimumab), but not when clinical efficacy failed to be achieved (guselkumab, ustekinumab, and CSF1R antagonist JNJ-40346527). Of note, RA M-DP scores have also been shown to decrease in recently diagnosed RA patients after an initial 6-months of conventional synthetic disease-modifying antirheumatic drugs (csDMARD) treatment. Accordingly, a pharmacodynamic (PD) M-DP score provides a biomarker-based test that objectively measures disease activity independent of clinical signs and symptoms. It has been discovered by the inventors of the present application that PD M-DP scores are significantly associated with clinical response to treatment, including with treatments not efficacious in the overall study population (ustekinumab and JNJ-40346527), and the PD M-DP scores can be used to predict the efficacy of a treatment, preferably before clinical efficacy is detected.

It has also been demonstrated that an M-DP panel with less than 14 analytes, such as a 4-analyte M-DP panel, performs at least as well as the originally-defined 14-analyte M-DP panel for PD composite scores, e.g., to: 1) significantly decrease after treatment with efficacious, but not with non-efficacious, therapies; and 2) be decreased significantly more in EULAR DAS28-CRP good vs. no response groups specifically with active treatments but not with placebo.

In certain embodiments of the application, RA M-DP scoring is utilized in small, early phase proof-of-mechanism studies to make relatively quick decisions about potential clinical efficacy or for interim futility analyses in larger phase 2 studies to make decisions about whether to continue to fully enroll the study or terminate the study early. Placebo comparator arms are not critical for the evaluations, allowing for reduced sample sizes. The estimated sample size needed per active treatment arm is small, for example, can be 10-15 patients. In another embodiment, RA M-DP scoring is used in platform or ‘pick-the-winner’ studies, in which treatments that do not significantly decrease M-DP scores are deprioritized.

### Biomarker Panels and Probes for Detecting Biomarkers

Developing biomarker-based tests (e.g., measuring cytokines) specific to the clinical assessment of RA has proved difficult in practice because of the complexity of RA biology—the various molecular pathways involved and the intersection of autoimmune dysregulation and inflammatory response. Adding to the difficulty of developing RA-specific biomarker-based tests are the technical challenges involved; e.g., the need to block non-specific matrix binding in serum or plasma samples, such as rheumatoid factor (RF) in the case of RA. The detection of cytokines using bead-based immunoassays, for example, is not reliable because of interference by RF; hence, RF-positive subjects cannot be tested for RA-related cytokines using this technology (and RF removal methods attempted did not significantly improve results). See S. Churchman et ah, Ann. Rheum. Dis. 2009, 68: A1-A56, Abstract A77. Approximately 70% of RA subjects are RF-positive, so any biomarker-based test that cannot assess RF-positive patients is obviously of limited use. Thus, it is difficult to develop a single test that can accurately and consistently assess, quantify, and monitor RA disease activity in every subject.

To achieve the maximum therapeutic benefits for individual subjects, it is important to be able to specifically quantify and assess the subject's disease activity at any particular time, determine the effects of treatment on disease activity, and predict future outcomes. No existing single biomarker or multi-biomarker test produces results demonstrating a high association with level of RA disease activity. The embodiments of the present teachings identify multiple serum biomarkers for the accurate clinical assessment of disease activity in subjects with chronic inflammatory disease, such as RA, along with methods of their use.

The biomarkers of use in the present disclosure include, for example, the following 14 biomarkers: annexin I (ANXA1), CXCL13 (C-X-C motif chemokine ligand 13, BLC, B lymphocyte chemoattractant), BPI (bactericidal/permeability-increasing protein), CRP (C-reactive protein), IL-6 (interleukin-6), CXCL10 (C-X-C motif chemokine ligand 10, IP-10, interferon-gamma induced protein 10), LEAP-1 (hepcidin, liver-expressed antimicrobial protein), MMP-1 (metalloproteinase-1), MMP-3 (metalloproteinase-3), PBEF (pre-B-cell colony-enhancing factor 1, NAMPT, nicotinamide phosphoribosyltransferase), PHI (phosphohexose isomerase, GPI, glucose-6-phosphate isomerase), SAA (serum amyloid A-1 protein, SAA1), SP-D (surfactant protein D), and TIMP-3 (tissue inhibitor of metalloproteinase 3, metalloproteinase inhibitor 3).

C-reactive protein (CRP), as noted above, is a protein synthesized by the liver in response to factors released by macrophages and fat cells (adipocytes). CRP levels in the blood increase when there is a condition causing inflammation somewhere in the body. A CRP test measures the amount of CRP in the blood to detect inflammation due to acute conditions or to monitor the severity of disease in chronic conditions. The standard CRP test measures high levels of the protein observed in diseases that cause significant inflammation. It measures CRP in the range from 8 to 1000 mg/L (or 0.8 to 100 mg/dL). In healthy adults, the normal concentrations of CRP vary between 0.8 mg/L to 3.0 mg/L. However, some healthy adults show elevated CRP at 10 mg/L. CRP concentrations also increase with age, possibly due to subclinical conditions. When there is an inflammatory stimulus, the CRP level can increase 10,000-fold from less than 50 μg/L to more than 500 mg/L.

Serum amyloid A1 protein, referred to herein as “SAA” and “SAA1,” is a protein made primarily in the liver. SAA circulates in low levels in the blood, and plays a role in the immune system. SAA may help repair damaged tissues, act as an antibacterial agent, and signal the migration of germ-fighting cells to sites of infection. Levels of this protein increase in the blood and other tissues under conditions of inflammation. SAA is also a major precursor of amyloid A fibril deposits in various tissues.

C-X-C Motif Chemokine Ligand 10, referred to interchangeably herein as “CXCL10,” “interferon gamma-induced protein 10,” “IP-10,” and “small-inducible cytokine B10,” is a pro-inflammatory chemokine that is involved in a wide variety of processes such as chemotaxis, differentiation, and activation of peripheral immune cells, regulation of cell growth, apoptosis and modulation of angiostatic effects. Mechanistically, binding of CXCL10 to the CXCR3 receptor activates G protein-mediated signaling and results in downstream activation of phospholipase C-dependent pathway, an increase in intracellular calcium production and actin reorganization (Smit MJ, et al. Blood 2003; 102:1959-1965; Gao J M, et al. Acta Pharmacol. Sin. 2009; 30: 193-201). In turn, recruitment of activated Th1 lymphocytes occurs at sites of inflammation (Smit MJ, et al. Blood 2003; 102:1959-1965; Cheeran M C, et al. J. Virol. 2003; 77: 4502-4515). CXCL10 is secreted by several cell types in response to IFN-γ.

Interleukin-6 (IL-6) is a cytokine with a wide variety of biological functions including inflammation and the maturation of B cells. The protein is primarily produced at sites of acute and chronic inflammation, where it is secreted into the serum and induces a transcriptional inflammatory response through interleukin 6 receptor, alpha (IL-6R). IL-6 is implicated in a wide variety of inflammation-associated disease states and autoimmune diseases, including susceptibility to diabetes mellitus and systemic juvenile rheumatoid arthritis.

C-X-C motif chemokine ligand 13, referred to interchangeably herein as “CXCL13,” “B cell attracting chemokine 1,” “BCA-1,” “B lymphocyte chemo-attractant,” and “BLC” is a small circulating cytokine that is chemotactic for B cells. CXCL13 exerts important functions in lymphoid neogenesis, and has been widely implicated in the pathogenesis of a number of autoimmune diseases and inflammatory conditions, as well as in lymphoproliferative disorders. CXCL13 elicits its effects by interacting with chemokine receptor CXCRS expressed on follicular B cells.

As used herein, “glucose-6-phosphate isomerase” or “GPI”, also known as “phosphohexose isomerase,” “PHI,” “phosphoglucose isomerase,” and “PGI” all refer to an enzyme secreted by lectin-stimulated T-cells that has different functions inside and outside the cell. In the cytoplasm, GPI interconverts glucose-6-phosphate (G6P) and fructose-6-phosphate (F6P). Extracellularly, GPI functions as a neurotrophic factor, or neuroleukin, that promotes survival of skeletal motor neurons and sensory neurons, and as a lymphokine that induces immunoglobulin secretion.

In some embodiments, provided herein are DNA-, RNA-, and protein-based diagnostic methods that either directly or indirectly detect the biomarkers described herein. The present invention also provides compositions, reagents, and kits for such diagnostic purposes. The diagnostic methods described herein may be qualitative or quantitative. Quantitative diagnostic methods may be used, for example, to compare a detected biomarker level to a cutoff or threshold level. Where applicable, qualitative or quantitative diagnostic methods can also include amplification of target, signal, or intermediary.

Any methods available in the art for measuring quantitative data for biomarkers, such as by detecting expression of biomarkers, are encompassed herein. The expression, presence, or amount of a biomarker of the invention can be detected on a nucleic acid level (e.g., as an RNA transcript) or a protein level. By “detecting or determining expression of a biomarker” is intended to include determining the quantity or presence of a protein or its RNA transcript for the biomarkers disclosed herein. Thus, “detecting expression” encompasses instances where a biomarker is determined not to be expressed, not to be detectably expressed, expressed at a low level, expressed at a normal level, or overexpressed.

In some embodiments, biomarkers are detected at the nucleic acid (e.g., RNA) level. For example, the amount of biomarker RNA (e.g., mRNA) present in a sample is determined (e.g., to determine the level of biomarker expression). Biomarker nucleic acid (e.g., RNA, amplified cDNA, etc.) can be detected/quantified using a variety of nucleic acid techniques known to those of ordinary skill in the art, including but not limited to, nucleic acid hybridization and nucleic acid amplification.

In some embodiments, a microarray is used to detect the biomarker. Microarrays can, for example, include DNA microarrays; protein microarrays; tissue microarrays; cell microarrays; chemical compound microarrays; and antibody microarrays. A DNA microarray, commonly referred to as a gene chip can be used to monitor expression levels of thousands of genes simultaneously. Microarrays can be used to identify disease genes by comparing expression in disease states versus normal states. Microarrays can also be used for diagnostic purposes, i.e., patterns of expression levels of genes can be studied in samples prior to the diagnosis of disease, and these patterns can later be used to predict the occurrence of a disease state in a healthy subject.

In some embodiments, the expression products are proteins corresponding to the biomarkers of the panel. In certain embodiments detecting the levels of expression products comprises exposing the sample to antibodies for the proteins corresponding to the biomarkers of the panel. In certain embodiments, the antibodies are covalently linked to a solid surface. In certain embodiments, detecting the levels of expression products comprises exposing the sample to a mass analysis technique (e.g., mass spectrometry).

In some embodiments, reagents are provided for the detection and/or quantification of biomarker proteins. The reagents can include, but are not limited to, primary antibodies that bind the protein biomarkers, secondary antibodies that bind the primary antibodies, affibodies that bind the protein biomarkers, aptamers (e.g., a SOMAmer) that bind the protein or nucleic acid biomarkers (e.g., RNA or DNA), and/or nucleic acids that bind the nucleic acid biomarkers (e.g., RNA or DNA). The detection reagents can be labeled (e.g., fluorescently) or unlabeled. Additionally, the detection reagents can be free in solution or immobilized.

In some embodiments, when quantifying the level of a biomarker(s) present in a sample, the level can be determined on an absolute basis or a relative basis. When determined on a relative basis, comparisons can be made to controls, which can include, but are not limited to historical samples from the same patient (e.g., a series of samples over a certain time period), level(s) found in a subject or population of subjects without the disease or disorder (e.g., RA), a threshold value, and an acceptable range.

Provided herein are isolated sets of probes capable of detecting a panel of biomarkers indicative of RA. In some embodiments, the isolated set of probes for detecting a panel of biomarkers consists of two, three, four, five, six, seven, eight, nine, ten, eleven, twelve, thirteen, or fourteen of biomarkers selected from the group consisting of: C-reactive protein pentraxin-related (CRP), serum amyloid A-1 protein (SAA), C-X-C motif chemokine 10 (CXCL10), C-X-C Motif Chemokine Ligand 13 (CXCL13), interleukin 6 (IL-6), phosphohexose isomerase (PHI), annexin I, BPI (bactericidal/permeability-increasing protein), LEAP-1 (liver-expressed antimicrobial protein), MMP-1 (metalloproteinase-1), MMP-3 (metalloproteinase-3), PBEF (pre-B-cell colony-enhancing factor 1), SP-D (surfactant protein D), and TIMP-3 (tissue inhibitor of metalloproteinase 3). In some embodiments, the isolated set of probes for detecting a panel of biomarkers consists of CRP, SAA, CXCL10, and at least one of CXCL13, IL-6 and PHI. In some embodiments, the panel of biomarkers consists of CRP, SAA, CXCL10 and CXCL13. In some embodiments, the panel of biomarkers consists of CRP, SAA, CXCL10 and IL-6. In some embodiments, the panel of biomarkers consists of CRP, SAA, CXCL10 and PHI.

Probes for use in the methods disclosed herein can be any molecule or agent that specifically detects a biomarker. In some embodiments, the probes include, but are not limited to, an aptamer (such as a slow-off rate modified aptamer (SOMAmer)), an antibody, an affibody, a peptide, and a nucleic acid (such as an oligonucleotide hybridizing to the gene or mRNA of a biomarker). An aptamer is an oligonucleotide or a peptide that binds specifically to a target molecule. An aptamer is usually created by selection from a large random sequence pool. Examples of aptamers useful for the invention include oligonucleotides, such as DNA, RNA or nucleic acid analogues, or peptides, that bind to a biomarker of the invention. In one embodiment, the aptamers are single-stranded DNA-based protein affinity binding reagents, such as SOMAmers developed by SomaLogic, Inc. (Boulder, Colo., USA). Under normal conditions (e.g., physiologic in serum), SOMAmers fold into specific shapes that bind target proteins with high affinity (sub-nM K a), but when SOMAmers are denatured, they can be detected and quantified by hybridizing to a standard DNA microarray. This dual nature of SOMAmers facilitates the detection of biomarkers that the SOMAmers specifically bind to. Kits

In some embodiments, a kit comprising the isolated set of probes capable of detecting a panel of biomarkers indicative of RA is provided.

Any of the compositions or probes can be provided in the form of a kit or a reagent mixture. By way of an example, labeled probes can be provided in a kit for the detection of a panel of biomarkers. Kits can include all components necessary or sufficient for assays, which can include, but is not limited to, detection reagents (e.g., probes with detectable markers), buffers, control reagents (e.g., positive and negative controls), amplification reagents, solid supports, labels, instruction manuals, etc. In some embodiments, the kit comprises a set of probes for the panel of biomarkers and a solid support to immobilize the set of probes. In some embodiments, the kit comprises a set of probes for the panel of biomarkers, a solid support, and reagents for processing the sample to be tested (e.g., reagents to isolate the protein or nucleic acids from the sample).

### Methods of Use

Provided herein are methods for predicting the efficacy of a treatment for rheumatoid arthritis (RA) or for monitoring the responsiveness to a treatment regimen in a subject being treated for RA. In some embodiments, the method comprises: (a) applying an isolated set of probes capable of detecting a panel of biomarkers indicative of RA to a biological sample to thereby measure quantitative data for each biomarker of the panel of biomarkers in the biological sample, wherein the biological sample is obtained from a subject in need of a treatment of RA at a time point T1 after the subject is treated with a therapy; (b) obtaining a treatment dataset comprising the quantitative data for all biomarkers of the panel of biomarkers measured in (a); (c) obtaining a baseline dataset comprising quantitative data for all biomarkers of the panel of biomarkers; (d) comparing the quantitative data in the treatment dataset with the corresponding quantitative data in the baseline dataset to obtain a change in each biomarker of the panel of biomarkers at the time point T1 after the subject is treated with the therapy; and (e) determining the molecular disease profile (M-DP) score of the subject at the time point T1 as the median value of the changes in all biomarkers of the panel of biomarkers measured in (d). The baseline dataset comprises quantitative data for all biomarkers of the panel of biomarkers measured from a subject not treated with the therapy. In a preferred embodiment, the baseline dataset comprises quantitative data for all biomarkers of the panel of biomarkers measured from a biological sample obtained from the subject before the subject is treated with the therapy. The baseline dataset can be measured before the subject is treated with the therapy and saved in the record for later use. The baseline dataset can also be measured from a stored biological sample obtained from the subject before the subject is treated with the therapy, together with the measurement of the treatment dataset.

In some embodiments, the M-DP score is determined as the median value of the log₂ transform of the ratio of the quantitative data for each biomarker in the treatment dataset over the corresponding quantitative data in the baseline dataset. In some embodiments, other math functions can be used for producing the M-DP, such as the mean value, the 25^{th} 75^{th} or other percentiles, of the log₂ transform of the ratio of the quantitative data for each biomarker in the treatment dataset over the corresponding quantitative data in the baseline dataset. Other transformations that act to provide an approximately normal distribution of the data, including but not limited to log 10, natural log, and square root transforms, can be used in place of log₂ transform of the ratios. Non-transformed ratios can be utilized when using median or other percentiles of the ratios to determine the M-DP score.

In some embodiments, the method further comprises (f) determining an M-DP score for the subject at at least one additional time point T2 after the subject is treated with the therapy; (g) determining a composite M-DP score for the subject as the mean value of the M-DP scores at the time point T1 and the at least one additional time point T2 after the subject is treated with the therapy; and (h) predicting the efficacy of the therapy in treating RA in the subject based on the composite M-DP score for the subject. For example, an M-DP score can be measured weekly, biweekly, triweekly or monthly after the subject is treated with the therapy, and a composite M-DP score based on the measured M-DP scores can be determined used to predict the efficacy of the therapy, preferably before a clinical efficacy is observed yet in the subject.

In some embodiments, the method further comprises (f) determining an M-DP score for each subject of a group of subjects in need of a treatment of RA at the time point T1 after the group of subjects are treated with the therapy; (g) determining a composite M-DP score for the group of subjects at the time point T1 as the mean value of the M-DP scores for all subjects in the group of subjects at the time point T1; and (h) predicting the efficacy of the therapy in treating RA based on the composite M-DP score.

In some embodiments, the group of subjects consists of 5 to 25 subjects. In some embodiments, the group of subjects consists of 10 to 20 subjects. In some embodiments, the group of subjects consists of 10 to 15 subjects, such as 10, 11, 12, 13, 14 or 15 subjects.

In some embodiments, the biological sample is selected from a tissue sample, a cellular sample, or a blood sample. In some embodiments, the biological sample is selected from a serum sample, a plasma sample, or a whole blood sample. In some embodiments, the biological sample is a serum sample from the subject.

In some embodiments, the panel of biomarkers consists of two, three, four, five or six of C-reactive protein pentraxin-related (CRP), serum amyloid A-1 protein (SAA), C-X-C motif chemokine 10 (CXCL10), C-X-C Motif Chemokine Ligand 13 (CXCL13), interleukin 6 (IL-6), and phosphohexose isomerase (PHI). In some embodiments, the panel of biomarkers consists of CRP, SAA, CXCL10 and at least one of CXCL13, IL-6 and PHI. In some embodiments, the panel of biomarkers consists of CRP, SAA, CXCL10 and CXCL13. In some embodiments, the panel of biomarkers consists of CRP, SAA, CXCL10 and IL-6. In some embodiments, the panel of biomarkers consists of CRP, SAA, CXCL10 and PHI.

Preferably, T1 is before a clinical efficacy of the therapy can be detected from the subject. In some embodiments, the time point T1 is about 4 to 12 weeks after the subject is treated with the therapy, including about 4 weeks, about 5 weeks, about 6 weeks, about 7 weeks, about 8 weeks, about 9 weeks, about 10 weeks, about 11 weeks, or about 12 weeks after the subject is treated with the therapy. In some embodiments, the time point T1 is about 4 to 8 weeks, such as 4, 5, 6, 7, or 8 weeks, or anytime in between, after the subject is treated with the therapy.

In some embodiments, the composite M-DP score is correlated with a clinical assessment. In some embodiments, the clinical assessment is selected from the group consisting of a DAS, a DAS28, a DAS28-CRP, a Sharp score, a tender joint count (TJC), a swollen joint count (SJC), Clinical Disease Activity Index (CDAI), and Simple Disease Activity Index (SDAI). In some embodiments, the clinical assessment is the DAS28-CRP.

In some embodiments, the efficacy of the therapy in treating RA is predicted based on the composite M-DP score before the efficacy is detected by a clinical assessment.

In some embodiments, the method further comprises continuing treating the subject(s) with the therapy, if the therapy is predicted to be effective. In some embodiments, the method further comprises stopping treating the subject(s) with the therapy, and/or treating the subject(s) with another therapy, if the therapy is predicted to be ineffective.

### Clinical Assessments

A method of the invention can further comprise clinically assessing RA disease activity in the subject. Clinical assessments of RA disease activity include measuring the subject's difficulty in performing activities, morning stiffness, pain, inflammation, and number of tender and swollen joints, an overall assessment of the subject by the physician, an assessment by the subject of how good s/he feels in general, and measuring the subject's erythrocyte sedimentation rate (ESR) and levels of acute phase reactants, such as CRP. Composite indices comprising multiple variables, such as those just described, have been developed as clinical assessment tools to monitor disease activity. The most commonly used are: American College of Rheumatology (ACR) criteria (DT Felson et al, Arth. Rheum. 1993, 36(6):729-740 and DT Felson et al, Arth. Rheum. 1995, 38(6):727-735); Clinical Disease Activity Index (CDAI) (D. Aletaha et al, Arth. Rheum. 2005, 52(9):2625-2636); the DAS (MLL Prevoo et al, Arth. Rheum. 1995, 38(1):44-48 and AM van Gestel et al, Arth. Rheum. 1998, 41(10): 1845-1850); Rheumatoid Arthritis Disease Activity Index (RADAI) (G. Stucki et al, Arth. Rheum. 1995, 38(6):795-798); and Simplified Disease Activity Index (SDAI) (JS Smolen et al, Rheumatology (Oxford) 2003, 42:244-257).

Current laboratory tests routinely used to monitor disease activity in RA subjects, such as CRP and ESR, are relatively non-specific (e.g., are not RA-specific and cannot be used to diagnose RA), and cannot be used to determine response to treatment or predict future outcomes. See, e.g., L. Gossec et al, Ann. Rheum. Dis. 2004, 63(6):675-680; EJA Kroot et al, Arth. Rheum. 2000, 43(8): 1831-1835; H. Makinen et al, Ann. Rheum. Dis. 2005, 64(10):1410-1413; Z. Nadareishvili et al., Arth. Rheum. 2008, 59(8): 1090-1096; NA Khan et al, Abstract, ACR/ARHP Scientific Meeting 2008; TA Pearson et al, Circulation 2003, 107(3):499-511; MJ Plant et al, Arth. Rheum. 2000, 43(7): 1473-1477; T. Pincus et al, Clin. Exp. Rheum. 2004, 22(Suppl. 35): S50-S56; and, PM Ridker et al, NEJM 2000, 342(12):836-843. In the case of ESR and CRP, RA subjects may continue to have elevated ESR or CRP levels despite being in clinical remission (and non-RA subjects may display elevated ESR or CRP levels). Some subjects in clinical remission, as determined by DAS, continue to demonstrate continued disease progression radiographically, by erosion.

Furthermore, some subjects who do not demonstrate clinical benefits still demonstrate radiographic benefits from treatment. See, e.g., FC Breedveld et al, Arth. Rheum. 2006, 54(1):26-37.

Accordingly, there is a need for clinical assessment tools that accurately assess an RA subject's disease activity level and that act as predictors of future course of disease.

Clinical assessments of disease activity contain subjective measurements of RA, such as signs and symptoms, and subject-reported outcomes, all difficult to quantify consistently. In clinical trials, the DAS is generally used for assessing RA disease activity. The DAS is an index score of disease activity based in part on these subjective parameters. Besides its subjectivity component, another drawback to use of the DAS as a clinical assessment of RA disease activity is its invasiveness. The physical examination required to derive a subject's DAS can be painful, because it requires assessing the amount of tenderness and swelling in the subject's joints, as measured by the level of discomfort felt by the subject when pressure is applied to the joints. Assessing the factors involved in DAS scoring is also time-consuming. Furthermore, to accurately determine a subject's DAS requires a skilled assessor so as to minimize wide inter—and intra-operator variability. A method of clinically assessing disease activity is needed that is less invasive and time-consuming than DAS, and more consistent, objective and quantitative, while being specific to the disease assessed (such as RA).

### Example

The present invention is further defined in the following Example. It should be understood that this Example, while indicating preferred embodiments of the invention, are given by way of illustration only. From the above discussion and this Example, one skilled in the art can ascertain the essential characteristics of this invention, and without departing from the spirit and scope thereof, can make various changes and modifications of the invention to adapt it to various uses and conditions.

### Methods

### Clinical Studies and Serum Samples

Serum samples and data from eight interventional clinical studies with seven different active treatments for RA or placebo treatment were obtained and analyzed: sirukumab (anti-IL-6) and placebo treatments in SIRROUND-M (NCT01689532; Takeuchi T, et al. Arthritis Res Ther. 2018 Mar. 7; 20(1): 42), SIRROUND-D (NCT01604343; Takeuchi T, et al. Ann Rheum Dis. 2017 Dec.; 76(12): 2001-2008), and SIRROUND-T (NCT01606761; Aletaha D, et al. Lancet. 2017 Mar. 25; 389(10075): 1206-1217); sirukumab and adalimumab (anti-TNF-alpha) treatments in SIRROUND-H (NCT02019472; Taylor PC, et al. Ann Rheum Dis. 2018 May; 77(5): 658-666); golimumab (anti-TNF-alpha) treatment in GO-FURTHER (NCT00973479; Weinblatt ME, et al. Ann Rheum Dis. 2013 March; 72(3): 381-9); guselkumab (anti-IL-23p19), ustekinumab (anti-IL-12/IL-23p40), and placebo treatments in CNTO1275ARA2001 (NCT01645280; Cox JD, et al. Lung Cancer. 1994 March; 10 Suppl 1: S161-6); JNJ-40346527 (colony-stimulating factor-1 inhibitor) and placebo treatments in 40346527ARA2001 (NCT01597739; Genovese MC, et al. J Rheumatol. 2015 October; 42(10): 1752-60). Plasma samples were obtained from the RA-MAP TACERA study (Cope AP, et al. Nat Rev Rheumatol. 2018 January; 14(1): 53-60) before (baseline) and 6-months after initiation of conventional synthetic disease-modifying anti-rheumatic drugs (csDMARDs). Details of treatment dose groups, concomitant methotrexate use, and previous experience with csDMARDs and biologics are reported in Table 1. The studies were conducted in accordance with the Declaration of Helsinki. The study protocols were reviewed and approved by an independent ethics committee or institutional review board for each study center. Written informed consent was obtained from all patients before study entry. Additional study details and patient characteristics are available in the referenced publications for each study. For SIRROUND-D and SIRROUND-T studies, sets of serum samples from demographically-matched healthy controls were obtained from commercial sources (BioIVT, Westbury, N.Y.).

The primary clinical outcome used for the current study was based on EULAR DAS28-CRP response criteria: good response, DAS28-CRP score <3.2 with a decrease >1.2; moderate response: DAS28-CRP score >3.2 with a decrease >1.2 or DAS28-CRP score <5.1 with a decrease >0.6 to 1.2 or; decrease in DAS28-CRP score <0.6 or DAS28-CRP score >5.1 with a decrease >0.6 to 1.2 (Wells G, et al. Ann Rheum Dis. 2009 June; 68(6): 954-60).

Additionally, a validation set of serum samples were commercially obtained (CERTAIN Biorepository (Pappas AD, et al. BMC Musculoskelet Disord. 2014 Apr. 1; 15:113), Coronna, LLC) for RA patients treated with rituximab (n=12) or abatacept (n=38), with serum samples obtained at baseline and at 3-months and 6-months of treatment. EULAR DAS28-CRP clinical response rates were 50% and 58% for good response, 32% and 33% for moderate response, and 18% and 8% no response, for rituximab and abatacept treatment groups, respectively.

### Somalogic SOMAscan

Serum samples from baseline (week 0) and the time points indicated in Table 1 were provided for quantification of 1189 serum analytes using the SomaScan v3.1 platform (SomaLogic, Boulder, Colo.; www.somalogic.com), with the exception of RA-MAP TACERA, for which plasma samples were analyzed for 1301 analytes using the SomaScan v3.2 platform. For some studies, a redacted dataset reporting data for a subset of the analytes was obtained. Relative fluorescence unit (RFU) data were sequentially normalized for hybridization controls (internal standards per samples), median signal across all samples (assumes same total protein concentration across sample set), and calibration controls (common sample standards across analysis plates). Samples failed quality control standards defined by Somalogic were excluded from analyses. The normalized RFU values were log₂-transformed for subsequent analyses.

TABLE 1
Description of studies

| | | | | Placebo- | csDMARD | bDMARD | Concominant | Serum |
|---|---|---|---|---|---|---|---|---|
| Study | Treatment | Dose (s) | Target | controlled | experience | experience | MTX | analysis |
| SIRROUND-M | Sirakumab | SC 100 mg q2w, SC 50 | IL−6 | No | MTX or SSZ | Naïve | No | Weeks 0, 4 |
| | | mg q4w | | | IR or INT | | | |
| SIRROUND-D | Sirakumab | SC 100 mg q2w, SC 50 | IL−6 | Yes | MTX or SSZ | Naïve/ | Yes or no | Weeks 0, 4, |
| | | mg q4w | | | IR or INT | not failed ^{b} | | 24 |
| SIRROUND-T | Sirakumab | SC 100 mg q2w, SC 50 | IL−6 | Yes | MTX IR or | TNFi IR | Yes or no | Weeks 0, 4, |
| | | mg q4w | | | INT | | | 24 |
| SIRROUND-H | Sirakumab | SC 100 mg q2w, SC 50 | IL−6 | No | MTX IR, INT, | Naïve | No | Weeks 0, 4 |
| | | mg q4w | | | or INAP | | | |
| SIRROUND-H | Adalimumab | SC 40 mg q2w | TNF-alpha | No | MTX IR, INT, | Naïve | No | Weeks 0, 4 |
| | | | | | or INAP | | | |
| GO-FURTHER | Golimumab | IV 2 mg/kg at Weeks, | TNF-alpha | Yes ^{a} | MTX IR (not | TNFi naïve | Yes | Weeks 0, 14 |
| | | 0 and 4 then q8w | | | INT) | | | |
| CNTO1275ARA2001 | Ustekinumab | SC 90 mg at Weeks 0 | IL-12/-23p40 | Yes | MTX IR (not | Naïve | Yes | Weeks 0, 4, |
| | | and 4, then q8w or q12w | | | INT) | | | 28 |
| CNTO1275ARA2001 | Guselkumab | SC 50 mg or SC 200 | IL−23pl9 | Yes | MTX IR (not | Naïve | Yes | Weeks 0, 4, |
| | | mg at Weeks 0 and 4, | | | INT) | | | 28 |
| | | then q8w | | | | | | |
| 40346527ARA2001 | JNJ-40346527 | oral 100 mg twice daily | CSF1-R | Yes | csDMARD IR | Naïve | Yes or no | Weeks 0, 4, |
| | | (BID) | | | | | | 12 |
| RA-MAP TACERA | csDMARDs † | as directed by | Various | No | csDMARD | Naïve | Yes or no ^{c} | Weeks 0, 26 |
| | | treating physician | | | naïve | | | |

^{a} Serum samples from placebo control group not analyzed with Somalogics SOMAscan platform
^{b} Patients who previously were treated with biologicals were permitted, as long as they had not failed anti-TNF or tocilizumab for safety or efficacy reasons and had not received biologicals within the past 3 months (6 weeks for etanercept or yisaipu and 4 weeks for anakinra).
^{c} Conventional DMARDs (no DMARDs prior to baseline visit): methotrexate ± hydrochloroqine (76%), hydrochloroquine only (9%), methotrexate + sulfasalizine/prednisone (8%), sulfasalazine only (4%), prednisone only (3%)
Abbreviations:
bDMARD, biologic disease-modifying anti-rheumatic drags;
csDMARDs, conventional synthetic disease-modifying anti-rheumatic drags;
INAP, inappropriate;
INT, intolerant;
IR, inadequate response;
MTX, methotrexate;
SSZ, sulfasalazine

### M-DP Score Calculation

Changes in analyte expression levels were calculated for individual samples as the log₂ transform of the ratio of the analyte level at the indicated time point (visit level) over the analyte level at baseline (baseline level) for a given subject, i.e., log 2(visit level/baseline level). Changes in RA disease-associated analytes per sample were summarized using a pharmacodynamic (PD) molecular disease profile (M-DP) score, defined as the median value of the log 2(visit level/baseline level) values for analytes in the defined M-DP analyte set. Analytes included in the M-DP were restricted to those associated with pre-treatment RA with high confidence, selected as those passing filter of FDR<0.05 and >1.5-ratio of geometric means of RA/healthy groups for an ‘up-regulated’ M-DP. PD M-DP scores in the log 2(visit level/baseline level) scale can be transformed to percent change from baseline, using the transformation: (2^{M-DP score}−1)×100%.

### MSD Assay Platform for 4-Analyte Panel

Using custom-designed Meso Scale Discovery (MS) U-PLEX panels, concentrations of CRP and SAA in panel one and CXCL10 and CXCL13 in panel 2, along with IL-6, were measured in the Coronna CERTAIN Biorepository serum samples from rituximab (Rituxan) and abatacept (Orencia)-treated RA patients. PD M-DP scores for the 4-analyte panel were calculated based on these concentrations of CRP, SAA, CXCL10, and CXCL13.

### Statistical Approaches

Statistical analyses were performed on log₂-transformed data, for either normalized Relative fluorescence unit (RFU) datasets or within-subject fold/baseline datasets. Significance of differences between groups was evaluated using General Linear Models, with t-tests when comparing two groups. Significance of difference from 0-change from baseline for log 2(visit level/baseline level) datasets was evaluated using 1-sample t-tests, testing difference from hypothesized mean of 0. Summary statistics in text are reported as mean±standard deviation unless otherwise indicated. Box & whiskers plots represent the median (line), interquartile range (box), and range (whiskers) of distributions, with symbols representing the values for each individual subject.

### Results

### Defining a Serum Molecular Disease Profile for RA

A baseline profile of serum analytes associated with RA populations was compared to demographically-matched healthy control populations. The RA populations included patients with moderate-to-severe disease activity who had inadequate response to, or were intolerant of, methotrexate (SIRROUND-D study, N=530) or TNF-inhibitor (TNFi) (SIRROUND-T study, N=321) treatment. At the time of the baseline visit for serum collection, the RA patients would have discontinued TNFi therapy for at least 3 months (or 6 weeks for the drug Etanercept or Yisaipu) and may or may not have maintained treatment with methotrexate (88% and 75% maintained methotrexate for SIRROUND-D and -T, respectively). Serum samples from 50 and 35 healthy control subjects were demographically-matched to the SIRROUND -D and -T RA populations, respectively.

Comparing levels of 379 analytes, measured with the Somalogic SOMAscan™ platform, in baseline-visit serum samples of RA patients (n=525 and 320 passing QC standards) to those in healthy controls (n=50 and 35), 34 and 24 analytes were considered significantly up-regulated and 7 and 8 down-regulated in disease in SIRROUND-D and -T studies, respectively (FDR<0.05 and |fold|>1.5) (data not shown). Among the commonly up-regulated analytes (>1.5-fold in one study and >1.45-fold in the other), 10 were intracellular proteins and 14 extracellular (secreted or membrane-anchored). All of the analytes significantly dysregulated in the TNFi-inadequate response (IR) population from the SIRROUND-T study were similarly differentially expressed in the csDMARD-IR population analyzed in the SIRROUND-D study. A molecular disease profile (M-DP) for RA was defined by the 14 commonly up-regulated extracellular analytes: annexin I, BLC (B lymphocyte chemoattractant, CXCL13), BPI (bactericidal permeability-increasing protein), CRP (C-reactive protein), IL-6, IP-10 (interferon-gamma induced protein 10, CXCL10), LEAP-1 (hepcidin), MMP-1 (metalloproteinase-1), MMP-3 (metalloproteinase-3), PBEF (NAMPT, nicotinamide phosphoribosyltransferase), PHI (GPI, glucose-6-phosphate isomerase), SAA (serum amyloid A-1 protein), SP-D (surfactant protein D), TIMP-3 (metalloproteinase inhibitor 3) (Table 2).

TABLE 2
Serum analytes associated with RA vs. healthy at baseline

| | GMean RA/healthy (p-value) | | | Entrez |
|---|---|---|---|---|
| Analyte ^{a} | SIRROUND-D | SIRROUND-T | TargetFullName | GeneSymbol |
| SAA | 4.72 (<0.0001) | 3.02 (0.0001) | Serum amyloid A-1 protein | SAA1 |
| LEAP-1 | 3.45 (<0.0001) | 2.84 (<0.0001) | Hepcidin | HAMP |
| | | | Pulmonary surfactant-associated | |
| SP-D | 2.49 (<0.0001) | 2.42 (<0.0001) | protein D | SFTPD |
| MMP-3 | 2.88 (<0.0001) | 2.40 (<0.0001) | Stromelysin-1 | MMP3 |
| BLC | 2.47 (<0.0001) | 1.92 (<0.0001) | C-X-C motif chemokine 13 | CXCL13 |
| IP-10 | 1.87 (<0.0001) | 1.76 (<0.0001) | C-X-C motif chemokine 10 | CXCL10 |
| CRP | 1.72 (<0.0001) | 1.70 (<0.0001) | C-reactive protein | CRP |
| TIMP-3 | 1.50 (<0.0001) | 1.69 (<0.0001) | Metalloproteinase inhibitor 3 | TIMP3 |
| PHI | 1.71 (<0.0001) | 1.66 (<0.0001) | Glucose-6-phosphate isomerase | GPI |
| | | | Nicotinamide | |
| PBEF | 1.54 (<0.0001) | 1.61 (<0.0001) | phosphoribosyltransferase | NAMPT |
| MMP-1 | 1.65 (<0.0001) | 1.60 (0.0002) | Interstitial collagenase | MMP1 |
| IL-6 | 1.66 (<0.0001) | 1.59 (<0.0001) | Interleukin-6 | IL6 |
| annexin 1 | 1.56 (<0.0001) | 1.55 (<0.0001) | Annexin A1 | ANXA1 |
| | | | Bactericidal permeability- | |
| BPI | 1.48 (0.0004) | 1.51 (0.0028) | increasing protein | BPI |

^{a} Serum analyte significantly associated with RA at baseline vs. healthy controls (FDR-BH < 0.05, GMean RA/healthy > 1.5 or < −1.33 in either SIRROUND-D [n =320, 49] or SIRROUND-T
^{b} Ratio of GMean of RA group over GMean of healthy control group (P-value RA vs. healthy), bolded when FDR < 0.05 and ratio > 1.5 or < −1.33

### Pharmacodynamic M-DP Scores

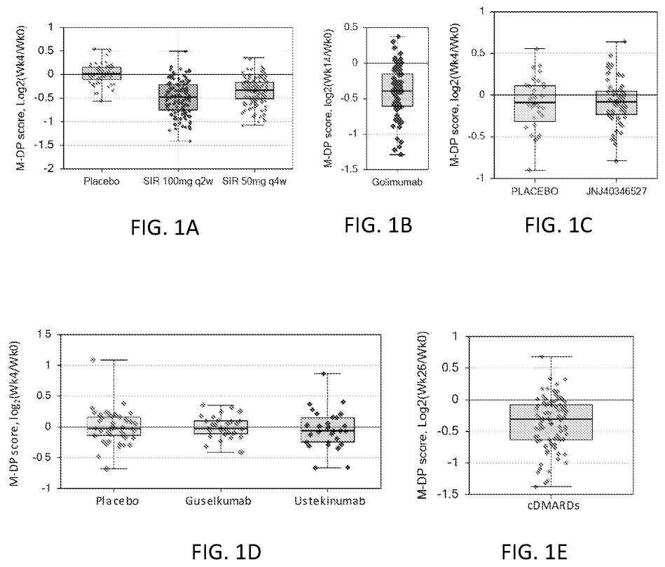

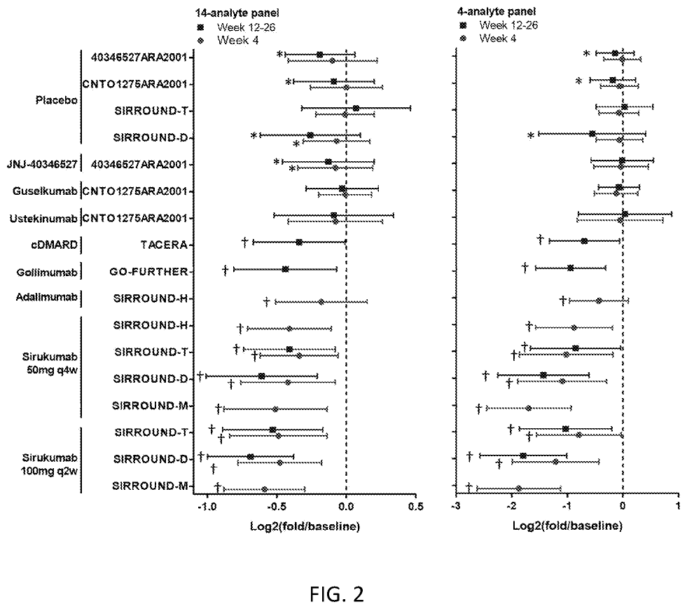

A composite score based on this 14-analyte molecular disease profile (M-DP) was calculated for each patient-sample as the median of the log₂ ratio of the analyte level at the indicated visit over the analyte level at baseline for the 14 analytes in the M-DP. In SIRROUND-D, this pharmacodynamic (PD) M-DP score (normalized to within-subject changes) was significantly decreased from baseline in the sirukumab 100 mg q2w and 50 mg q4w groups, relative to placebo, at week 4 in SIRROUND-D (Mean±SD log 2(week 4/baseline) scores of -0.48±0.30 and -0.42±0.34, translated as GMean 25% and 21% decreases, respectively) and -T (GMean 29% decrease and 22% decreases, respectively) and maintained through week 24 (FIG. 1, FIG. 2, and Table 3). The M-DP scores at week 4 decreased from baseline by at least one standard deviation from the group mean in these two studies and in two additional phase 3 studies for sirukumab treatment (SIRROUND-M, SIRROUND-H), with coefficient of variation of 22% (GMean; range 20-26%) (Table 3).

In other clinical studies in which SOMAscan data was available, GMean M-DP scores were not decreased by guselkumab (anti-IL-23p19), ustekinumab (anti-IL-12/-23p40), or the CSF1R-inhibitor JNJ-40346527 at either week 4 or the latest time point evaluated (weeks 28, 28, and 12, respectively) (Table 3). Significant M-DP scores of at least one standard deviation below zero were observed with 14-weeks treatment with the TNFi golimumab IV (26% decrease, GO-FURTHER study, week 4 evaluation not available) but not with 4-week treatment with adalimumab SC 40 mg q2w (12% decrease, p<0.0001; SIRROUND-H study, later time points not available) (Table 3). A significant M-DP score of at least one standard deviation below zero was also observed with 6-month csDMARD treatment of recently diagnosed RA cases (Table 3).

TABLE 3
Pharmacodynamics and response association of 14-analyte M-DP scores

| | M-DP | | M-DP score (log2 (fold/baseline): mean ± SD [N] ^{a} | | | | Mean difference ± | |
|---|---|---|---|---|---|---|---|---|
| | score at | | | DAS28-CRP response group ^{c} | | | SD (p-value) ^{d} | Response |
| Study | week ^{a} | Treatment group ^{b} | All treated | Good | Moderate | No | Good vs. No | at week ^{c} |
| SIRROUND-M | 4 | Sirukumab 100 | −0.59 ± 0.29 | −0.55 ± 0.27 | −0.70 ± 0.34 | −0.51 ± 0.10 [2] | n/a | 24 |
| | | mg q2w | [61]+ | [44]+ | [15]+ | | | |
| | | Sirukumab 50 | −0.51 ± 0.37 | −0.59 ± 0.38 | −0.46 ± 0.30 | −0.19 ± 0.25 [7] | −0.40 ± 0.37 | 24 |
| | | mg q4w | [61]+ | [41]+ | [13]* | | (0.0100) | |
| SIRROUND-D | 4 | Sirukumab 100 | −0.48 ± 0.30 | −0.50 ± 0.29 | −0.51 ± 0.30 | −0.33 ± 0.32 [28]+ | −0.27 ± 0.30 | 24 |
| | | mg q2w | [205]+ | [116]+ | [61]+ | | (0.0056) | |
| | | Sirukumab 50 | −0.42 ± 0.34 | −0.44 ± 0.32 | −0.43 ± 0.36 | −0.36 ± 0.35 [44]+ | −0.08 ± 0.33 | 24 |
| | | mg q4w | [201]+ | [98]+ | [59]+ | | (0.1800) | |
| | | Placebo | −0.07 ± 0.24 | −0.08 ± 0.32 | −0.11 ± 0.23 | −0.05 ± 0.21 [62] | −0.03 ± 0.25 | 24 |
| | | | [118]* | [27] | [29] * | | (0.6400) | |
| | 24 | Sirukumab 100 | −0.69 ± 0.31 | −0.69 ± 0.34 | −0.75 ± 0.20 | −0.52 ± 0.37 [2] | n/a | 24 |
| | | mg q2w | [27]+ | [20]+ | [5]* | | | |
| | | Sirukumab 50 | −0.61 ± 0.40 | −0.65 ± 0.39 | −0.59 ± 0.39 | 0.06 ± 0.00 [1] | n/a | 24 |
| | | mg q4w | [28]+ | [20]+ | [7]* | | | |
| | | Placebo | −0.26 ± 0.36 | −0.13 ± 0.22 | −0.17 ± 0.26 | −0.40 ± 0.45 [14] * | 0.27 ± 0.37 | 24 |
| | | | [33] * | [11] | [8] | | (0.0847) | |
| SIRROUND-T | 4 | Sirukumab 100 | −0.49 ± 0.35 | −0.55 ± 0.35 | −0.54 ± 0.35 | −0.35 ± 0.33 [39]+ | −0.20 ± 0.34 | 24 |
| | | mg q2w | [134]+ | [65]+ | [30]+ | | (0.0042) | |
| | | Sirukumab 50 | −0.34 ± 0.28 | −0.35 ± 0.30 | −0.37 ± 0.26 | −0.29 ± 0.26 [30]+ | −0.06 ± 0.29 | 24 |
| | | mg q4w | [127]+ | [63]+ | [34]+ | | (0.3128) | |
| | | Placebo | −0.01 ± 0.21 | −0.02 ± 0.18 | −0.07 ± 0.22 | 0.05 ± 0.24 [29] | −0.07 ± 0.22 | 24 |
| | | | [56] | [24] | [3] | | (0.2148) | |
| | 24 | Sirukumab 100 | −0.53 ± 0.36 | −0.59 ± 0.38 | −0.54 ± 0.37 | −0.41 ± 0.28 [40]+ | −0.18 ± 0.35 | 24 |
| | | mg q2w | [136]+ | [65]+ | [31]+ | | (0.0105) | |
| | | Sirukumab 50 | −0.41 ± 0.33 | −0.46 ± 0.35 | −0.41 ± 0.29 | −0.34 ± 0.31 [31]+ | −0.12 ± 0.34 | 24 |
| | | mg q4w | [128]+ | [63]+ | [34]+ | | (0.1135) | |
| | | Placebo | 0.07 ± 0.39 | −0.11 ± 0.27 | −0.25 ± 0.59 | 0.25 ± 0.37 [29] * | −0.36 ± 0.33 | 24 |
| | | | [56] | [24] | [3] | | (0.0003) | |
| SIRROUND-H | 4 | Sirukumab 50 | −0.41 ± 0.30 | −0.45 ± 0.33 | −0.40 ± 0.30 | −0.38 ± 0.22 [12]+ | −0.07 ± 0.30 | 24 |
| | | mg q4w | [96]+ | [29]+ | [54]+ | | (0.5231) | |
| | | Adalimumab 40 | −0.18 ± 0.33 | −0.22 ± 0.37 | −0.17 ± 0.33 | −0.14 ± 0.26 [23] * | −0.06 ± 0.33 | 24 |
| | | mg q2w | [100] + | [34] * | [43] * | | (0.3890) | |
| GO-FURTHER | 14 | Golimumab IV | −0.44 ± 0.37 | −0.56 ± 0.39 | −0.45 ± 0.34 | −0.21 ± 0.27 [22] * | −0.35 ± 0.35 | 24 |
| | | | [100]+ | [40]+ | [38]+ | | (0.0004) | |
| CNTO1275ARA2001 | 4 | Ustekinumab | −0.08 ± 0.34 | −0.24 ± 0.34 | −0.04 ± 0.29 | 0.08 ± 0.31 [31] | −0.32 ± 0.32 | 24 |
| | | | [36] | [14] * | [9] | | (0.0367) | |
| | | Guselkumab | −0.01 ± 0.19 | −0.04 ± 0.19 | 0.02 ± 0.20 | −0.04 ± 0.19 [12] | 0.00 ± 0.19 | 24 |
| | | | [31] | [7] | [12] | | (0.9362) | |
| | | Placebo | 0.00 ± 0.26 | 0.02 ± 0.40 | 0.01 ± 0.15 | −0.01 ± 0.30 [26] | 0.03 ± 0.32 | 24 |
| | | | [50] | [5] | [19] | | (0.8116) | |
| | 28 | Ustekinumab | −0.09 ± 0.43 | −0.34 ± 0.33 | −0.05 ± 0.23 | −0.15 ± 0.49 [13] | −0.49 ± 0.41 | 24 |
| | | | [36] | [14]* | [9] | | (0.0288) | |
| | | Guselkumab | −0.03 ± 0.26 | −0.08 ± 0.10 | 0.01 ± 0.34 | −0.05 ± 0.25 [12] | −0.03 ± 0.21 | 24 |
| | | | [31] | [7] | [12] | | (0.8276) | |
| | | Placebo | −0.09 ± 0.29 | 0.02 ± 0.40 | 0.01 ± 0.15 | −0.01 ± 0.30 [26] | 0.04 ± 0.32 | 24 |
| | | | [50] * | [5] | [19] | | (0.8360) | |
| 40346527ARA2001 | 4 | JNJ-40346527 | −0.08 ± 0.27 | 0.29 ± 0.42 | −0.05 ± 0.48 | −0.09 ± 0.54 [23] | 0.08 ± 0.52 | 12 |
| | | | [61] * | [5] | [28] | | (0.5900) | |
| | | Placebo | −0.10 ± 0.32 | −0.02 ± 0.55 | −0.07 ± 0.28 | 0.10 ± 0.26 [10] | −0.15 ± 0.39 | 12 |
| | | | [31] | [6] | [14] | | (0.3100) | |
| | 12 | JNJ-40346527 | −0.13 ± 0.33 | −0.50 ± 0.61 | −0.07 ± 0.55 | 0.18 ± 0.50 [22] | −0.4210.52 | 12 |
| | | | [56] * | [6] | [27] | | (0.0094) | |
| | | Placebo | −0.19 ± 0.25 | −0.06 ± 0.49 | −0.18 ± 0.33 | −0.15 ± 0.28 [10] | 0.0910.37 | 12 |
| | | | [30] * | [6] | [14] | | (0.4400) | |
| TACERA | 26 | csDMARDs | −0.3410.33 | −0.5010.36 | −0.3910.39 | −0.0610.31 [22] | −0.54 ± 0.35 | 26 |
| | | | [95]+ | [50]+ | [23]+ | | (<0.0001) | |

^{a} M-DP scores evaluated at indicated week, reported as log2 (fold/baseline), for 14-analyte M-DP panel.
* p < 0.05 and † p < 0.0001 vs. baseline (paired t-test).
Bolded when absolute value of mean M-DP score >= SD.
^{b} Sirukumab 100 mg q2w, Sirukumab subcutaneous 100 mg q2 weeks; Sirukumab 50 mg q4w, Sirukumab subcutaneous 50 mg q4 weeks;
Golimumab IV, Golimumab intravenous 2 mg/kg at Weeks 0 and 4, then q8 weeks thereafter plus concomitant weekly methotrexate;
Ustekinumab, Ustekinumab subcutaneous 90 mg at Weeks 0, 4, then q8 weeks, ustekinumab sc 90 mg at Weeks 0, 4, then every 12 (q 12) weeks;
Guselkumab, Guselkumab sc 200 mg at Weeks 0, 4, then q8 weeks, or guselkumab sc 50 mg at Weeks 0, 4, then q8 weeks;
JNJ-40346527, JNJ-40346527 oral 100 mg twice daily (BID);
csDMARDs, Conventional synthetic DMARDs (no DMARDs prior to baseline visit): hydrochloroquine only (9%), methotrexate ± hydrochloroqine (76%), methotrexate + sulfasalizine/prednisone (8%), sulfasalazine only (4%), prednisone only (3%)
^{c} DAS28-CRP response evaluated at the week indicated in column “Response at week”
^{d} Difference of group means ± standard devation (p-value for comparison) between DAS28-CRP EULAR good vs. no response groups for log2 (fold/baseline) values of 14-analyte M-DP scores. Bolded when p < 0.05.

Overall, treatments that were clinically efficacious in the underlying clinical studies (sirukumab, golimumab, and csDMARDs), but not those that were not clinically efficacious (ustekinumab, guselkumab, and JNJ-40346527), had significant M-DP scores of at least one standard deviation below zero in magnitude, corresponding to greater than 20% decreases. The exception was for adalimumab treatment, which had a significant decrease of only 12% (p<0.0001), but only week 4 M-DP scores were available and not later time points. For placebo treatment, PD M-DP scores had less than one standard deviation below zero for all studies, with the largest week 4 and week 12-28 decreases being 7% and 16%, respectively.

Intercorrelations of log 2(week 4/baseline) values among the 14 analytes in the M-DP were on average modest, with R_{Sp} in SIRROUND-T study averaging 0.22±0.18 (SD), ranging from −0.11 to 0.83. Annexin-I, BPI, and SP-D were the most highly intercorrelated analytes (correlations from 0.74 to 0.83). Correlations between the composite M-DP score and these 14-analytes ranged from 0.09 (IP-10/CXCL10) and 0.64 (BPI), with a correlation with CRP of 0.51 (Table 51).

TABLE S1
Correlations between M-DP scores and the 14 M-DP analytes

| | baseline/ | |
|---|---|---|
| Analyte ^{a} | healthy | Week 4/baseline |
| annexin I | 0.54 | 0.58 |
| CXCL13/BLC | 0.60 | 0.42 |
| BPI | 0.42 | 0.64 |
| CRP | 0.45 | 0.51 |
| IL-6 | 0.59 | 0.56 |
| CXCL10/IP-10 | 0.48 | 0.09 |
| LEAP-1/hepcidin | 0.42 | 0.47 |
| MMP-1 | 0.41 | 0.63 |
| MMP-3 | 0.65 | 0.40 |
| PBEF | 0.29 | 0.35 |
| PHI | 0.68 | 0.43 |
| SAA | 0.53 | 0.58 |
| SP-D | 0.51 | 0.62 |
| TIMP-3 | 0.34 | 0.52 |

^{a} Spearman coefficient of correlation of ranks between 14-analyte M-DP score and indicated analyte for dataset of baseline values of RA subjects normalized to mean of healthy control set (baseline/healthy) or for SIRROUND-T dataset of within-subject week 4/baseline values (Week 4/baseline)

### Clinical Response Associations

Week 4 PD M-DP scores were significantly associated with week 24 clinical response for sirukumab 100 mg q2w treatment, but not consistently for the 50 mg q4w dosing regimen (p<0.05 for EULAR DAS28-CRP good vs. no response) (Table 3). Week 24 clinical response associations with ustekinumab, but not guselkumab, treatment were observed for both week 4 and week 28 PD M-DP scores. Week 12 clinical response associations with JNJ-40346527 treatment were observed only for week 12 but not week 4 PD M-DP scores. Significant associations of clinical response with PD M-DP scores were also observed for 14-week golimumab IV and 26-week csDMARD treatments, with week 4 M-DP scores not available.

There were no significant associations of clinical response to week 4 M-DP scores for placebo treatment in any of the studies (Table 3). There was an association of clinical response with week 12-28 M-DP scores for placebo treatment in only 1 of 4 studies (SIRROUND-T), but the association was driven by a significant positive PD M-DP score in the no response group (19% increase, p=0.0011) rather than a negative score in good response group (7% decrease, p=0.058) (Table 3).

Despite the treatment-specific significant associations with EULAR DAS28-CRP response, the distributions between good and no response groups had substantial overlap. Therefore, the PD M-DP scores calculated from a population of subjects would not have practical predictive power to act as predictors (e.g., for week 4 M-DP scores) or surrogates (for week 12-28 M-DP scores) of clinical response at the individual subject level.

Exclusion of any one single analyte from the M-DP panel of 14 analytes did not appreciably impact the pharmacodynamic changes in M-DP scores nor clinical response associations (data not shown). This included the exclusion of CRP, for which clinical response associations trended towards greater significance for active treatment groups when excluded.

### Permutations of Analyte Set

To enable practical implementation of an assay platform to evaluate M-DP scores with minimal blood volumes, cost, and technical ease, the ability to define a minimal panel of analytes for the M-DP that retained the PD associations with clinical efficacy and DAS28-CRP response was evaluated.

There are 16,383 possible panel combinations of the 14 M-DP analytes for panels including from 1 to all 14 analytes. Considering a nominal p-value of 0.05 for clinical response association, the family-wise error rate under the overly conservative assumption that each panel is independent would be 3.0×10⁻⁶. An effective p-value of 3.1*10⁻⁷ would result if 5 studies with p=0.05 were combined using the Fisher's combined probability test.

In selecting the best minimal set of analytes that performs similarly to the full 14-analyte set, analyte sets that results in significant associations between DAS28-CRP EULAR good vs. no response with p<0.05 for each study were prioritized (Table 4). Week 4 PD M-DP scores for the analyte sets were used to test for association of week 24 clinical response for the 4 SIRROUND studies with sirukumab SC 50 mg q4w treatment and week 14 changes for golimumab IV treatment (GO-FURTHER study). Association with 6-month clinical response was tested for 6-month methotrexate treatment (RA-MAP TACERA study). Because significant associations with clinical response were observed only at later time points for ustekinumab (CNT01275ARA2001 study) and JNJ-40346527 (40346527ARA2001 study), the week 28 and 12 changes were tested for association with week 24 and week 12 clinical response, respectively. 29 combinations of analytes met these criteria, ranging from 4 to 8 analytes.

These 29 panels were then ranked by the mean of the difference between DAS28-CRP good vs. no response groups in M-DP scores calculated using the analytes in the model. The top 3 models each included 4 analytes: 1) CRP+CXCL10+IL-6+SAA; 2) CRP+CXCL10+CXCL13+SAA; and CRP+CXCL10+PHI+SAA, with median increases of at least 48% for the 4 analytes. These 3 panels retained the top performance when the rankings excluded results from the sirukumab studies, from which the original 14-analyte M-DP panel was defined. The 4-analyte panel of CRP+CXCL10+CXCL13+SAA was selected for further evaluation among the 3. Intercorrelations of log 2(week 4 test level/baseline level) values among CRP, CXCL10, CXCL13, and SAA in the SIRROUND-T study ranged from non-correlated (CXCL10 with CRP and SAA: R_{Sp} of −0.05 and −0.03), weakly correlated (CXCL13 with CRP, CXCL10, and SAA: R_{Sp} of 0.22, 0.30, and 0.28, respectively) to moderately correlated (CRP with SAA: R_{Sp} of 0.58) (Table S2). Correlations between these 4-analytes and the composite 4-analyte PD M-DP score were 0.15, 0.50, 0.67, and 0.89 for CXCL10, CXCL13, CRP, and SAA, respectively (Table S2).

TABLE S2
Intercorrelations between 4-analyte M-DP score and analytes

| Log2(week 4/baseline) | | R(Sp) |
|---|---|---|
| M-DP score | SAA | 0.89 |
| | CRP | 0.67 |
| | CXCL13/BLC | 0.50 |
| | CXCL10/IP-10 | 0.15 |
| SAA | CRP | 0.58 |
| | CXCL13/BLC | 0.28 |
| | CXCL10/IP-10 | −0.03 |
| CRP | CXCL13/BLC | 0.22 |
| | CXCL10/IP-10 | −0.05 |
| CXCL13/BLC | CXCL10/IP-10 | 0.30 |

^{a} Spearman coefficient of correlation of ranks between 4-analyte M-DP score/indicated analyte for SIRROUND-T dataset of within-subject week 4/baseline values (Week 4/baseline)

### 4-Analyte M-DP Score PD and Clinical Response Associations

Analogous to that observed for the 14-analyte M-DP panel, PD M-DP scores for the 4-analyte M-DP panel of CRP, CXCL10, CXCL13, and SAA were significantly below zero by at least one standard deviation, corresponding to greater than 60% decrease, for efficacious treatments (sirukumab, golimumab, and csDMARDs), but not for treatments that were not clinically efficacious (ustekinumab, guselkumab, and JNJ-40346527) (Table 5). Analogously, the exception was for adalimumab treatment, which had a significant decrease of 26% (p<0.0001), but only week 4 M-DP scores were available and not later time points. For placebo treatment, PD M-DP scores were less than one standard deviation below zero for all studies, with the largest week 4 and week 12-28 decreases being 5% and 32%, respectively (Table 5).

Week 4 PD M-DP scores were significantly associated with week 24 clinical response for sirukumab 100 mg q2w and 50 mg q4w treatments in SIRROUND-M, -D, and -T (p<0.05 for EULAR DAS28-CRP good vs. no response) and similarly for sirukumab 50 mg q4w (p=0.055) and adalimumab (p=0.022) treatments in SIRROUND-H (Table 5). Week 24 clinical response associations with ustekinumab, but not guselkumab, treatment were observed for both week 4 and week 28 PD M-DP scores (Table 5). Week 12 clinical response associations with JNJ-40346527 treatment were observed only for week 12 but not week 4 PD M-DP scores (Table 5). Significant associations of clinical response with PD M-DP scores were also observed for 14-week golimumab IV and 26-week csDMARD treatments, with week 4 M-DP scores not available (Table 5).

There were no significant associations of clinical response to week 4 M-DP scores for placebo treatment in any of the studies (Table 5). There was an association of clinical response with week 12-28 M-DP scores for placebo treatment in only 1 of 4 studies (SIRROUND-T), but the association was driven by a significant positive PD M-DP score in the no response group (17% increase, p=0.010) rather than a negative score in the good response group (10% decrease, p=0.12) (Table 5).

### Validation of 4-Analyte M-DP Score for Abatacept and Rituximab Treatments

The 4-analyte panel of CRP, SAA, CXCL10, and CXCL13 was measured by MSD U-PLEX assays in serum samples from 38 abatacept- and 12 rituximab-treated RA patients at baseline and 3—and 6—month visits. These samples were not previously evaluated for the M-DP panel development or reduction. Decreases in M-DP scores concurred with the clinical efficacy observed for abatacept treatment, with significant PD M-DP scores at 3—and 6—months for abatacept (GMean 45% decrease, p<0.0001 for both) (Table 6). These PD M-DP scores did not reach the 1-standard deviation below zero and 60% decrease threshold observed for sirukumab and golimumab but were greater than the 26% decrease (0.8-standard deviations below zero) observed for adalimumab at week 4. Significant M-DP scores were also observed for rituximab at 6-months (GMean 36% decrease, p=0.040), with a trend for scores below zero at 3-months (GMean 30% decrease, p=0.17) (Table 6).

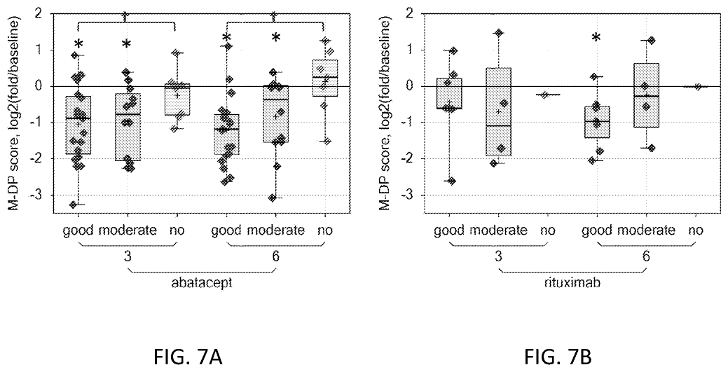

The 3—and 6—month PD M-DP scores were significantly associated with week 24 clinical response for abatacept (p<0.05 for EULAR DAS28-CRP good vs. no response), with 51%, 49%, and 16% GMean decreases at month 3 in good, moderate, and no response groups, respectively (Table 6, FIG. 7). For rituximab treatment, only 1 patient was in the no response group so associations of good vs. no response could not be made.

These results with abatacept and rituximab treatment confirm that significant decreases in M-DP scores occur with clinically efficacious treatments, extending the utility to biologic treatments that do not target cytokines but rather immune cells. However, magnitude of the decrease may be smaller and time required longer for some treatments, like observed for rituximab.

Therefore, the 4-analyte M-DP panel has been demonstrated to perform at least as well as the originally-defined 14-analyte M-DP panel for PD composite scores to: 1) significantly decrease after treatment with efficacious, but not with non-efficacious, therapies; and 2) be decreased significantly more in EULAR DAS28-CRP good vs. no response groups specifically with active treatments but not with placebo.

### Correlations Between Change in Clinical Disease Activity and M-DP Scores

Changes in DAS28-CRP scores evaluated at weeks 12-26 were moderately correlated with PD M-DP scores at weeks 12-28, calculated from either the 14—or 4—analyte panel, for active treatment groups, excluding guselkumab (Pearson's correlation coefficient R from 0.31 to 0.60, excluding SIRROUND-D sirukumab 100 mg q2w at week 24, with r=0.19-0.20) (Table 7). Correlations between changes in DAS28-CRP scores evaluated at weeks 12-26 and week 4 PD-M-DP scores for active treatment groups were generally weaker compared to the correlations with week 12-28 M-DP scores (R from 0.13 to 0.50) (Table 7). Correlations of changes in DAS28-CRP scores and PD M-DP scores for placebo treatment groups were variable, ranging from R of 0.00 to 0.60, likely in part due to variability in the dynamic range of changes in DAS28-CRP scores between studies (Table 7). Correlations for the guselkumab treatment group were weak (R=0.06 to 0.30), with limited dynamic range for changes in DAS28-CRP scores (Table 7). The strength of correlations was generally similar whether using the 14—or 4-analyte panel to calculate M-DP scores.

TABLE 4
Selection of minimal panel for M-DP scoring

| | | | | | | | | | | | | | Difference, Geo.Mean ^{b} | |
|---|---|---|---|---|---|---|---|---|---|---|---|---|---|---|
| | Analytes included in panel (marked ‘Y’) | | | | | | | | | | | Num | All | Excluding SIR |
| Model ^{a} | SAA | IL6 | IP10 | CRP | TIMP3 | PHI | LEAP1 | MMP1 | BLC | MMP3 | annexin-1 | analytes | studies | studies ^{c} |
| 1218 | Y | Y | Y | Y | — | — | — | — | — | — | — | 4 | 0.63 | 0.57 |
| 1186 | Y | — | Y | Y | — | — | — | — | Y | — | — | 4 | 0.58 | 0.56 |
| 418 | Y | — | Y | Y | — | Y | — | — | — | — | — | 4 | 0.57 | 0.58 |
| 1258 | Y | — | Y | Y | Y | — | — | — | — | — | — | 4 | 0.51 | 0.46 |
| 481 | Y | Y | Y | — | — | — | — | — | Y | — | — | 4 | 0.48 | 0.56 |
| 1326 | Y | Y | — | — | — | Y | Y | Y | Y | — | — | 6 | 0.47 | 0.56 |
| 3118 | Y | Y | Y | Y | — | — | — | Y | — | Y | — | 6 | 0.45 | 0.52 |
| 421 | Y | Y | — | — | Y | Y | — | — | — | — | — | 4 | 0.45 | 0.55 |
| 1230 | Y | Y | Y | — | — | — | — | Y | — | — | — | 4 | 0.45 | 0.52 |
| 3245 | Y | Y | Y | Y | — | — | — | Y | Y | — | — | 6 | 0.45 | 0.52 |
| 1441 | Y | Y | — | — | Y | Y | Y | — | Y | — | — | 6 | 0.44 | 0.54 |
| 3233 | Y | Y | Y | — | — | — | Y | Y | Y | — | — | 6 | 0.44 | 0.52 |
| 1518 | Y | Y | Y | Y | — | Y | — | Y | — | — | — | 6 | 0.44 | 0.51 |
| 3532 | Y | Y | Y | Y | — | Y | Y | Y | Y | — | — | 8 | 0.44 | 0.52 |
| 3553 | Y | Y | Y | Y | — | Y | Y | Y | — | Y | — | 8 | 0.43 | 0.50 |
| 3277 | Y | Y | Y | Y | Y | — | — | — | Y | — | — | 6 | 0.43 | 0.48 |
| 1189 | Y | Y | Y | — | Y | — | — | — | — | — | — | 4 | 0.42 | 0.49 |
| 3491 | Y | Y | Y | Y | Y | Y | Y | — | — | Y | — | 8 | 0.42 | 0.48 |
| 3563 | Y | Y | — | Y | Y | Y | Y | Y | — | Y | — | 8 | 0.42 | 0.48 |
| 3493 | Y | Y | Y | Y | Y | Y | Y | — | Y | — | — | 8 | 0.42 | 0.49 |
| 1506 | Y | Y | Y | Y | Y | Y | — | — | — | — | — | 6 | 0.42 | 0.48 |
| 1446 | Y | Y | Y | — | Y | Y | Y | — | — | — | — | 6 | 0.41 | 0.48 |
| 3496 | Y | Y | Y | — | Y | Y | Y | Y | Y | — | — | 8 | 0.40 | 0.51 |
| 1516 | Y | Y | Y | — | Y | Y | — | Y | — | — | — | 6 | 0.40 | 0.51 |
| 3565 | Y | Y | — | Y | Y | Y | Y | — | — | Y | Y | 8 | 0.40 | 0.45 |
| 7402 | Y | Y | Y | Y | Y | — | — | Y | Y | Y | — | 8 | 0.39 | 0.47 |
| 3298 | — | Y | Y | Y | Y | — | Y | — | Y | — | — | 6 | 0.38 | 0.42 |
| 3310 | — | Y | Y | — | Y | — | Y | Y | Y | — | — | 6 | 0.36 | 0.44 |
| 5580 | Y | Y | Y | — | Y | Y | Y | Y | — | — | Y | 8 | 0.34 | 0.43 |

^{a} Models from panels including the analytes marked “Y” are reported only if p < 0.05 for DAS28-CRP good vs. no response comparisons of M-DP scores calculated for the analytes in the model for all studies evaluated (SIRROUND-M, -D, and -T for sirukumab 50 mg q4w at Wk4; GO-FURTHER for golimumab at Wkl4; CNTO1275ARA2001-ustekinumab at Wk28; 40346527ARA2001-JNJ-40346527 at Wk12; RA-MAP TACERA-csDMARDs at Wk26).DAS28-CRP response based on visit reported in Table 3. BPI, PBEF, and SP-D not shown because not included in panels for the reported models.
^{b} The mean of the difference between DAS28-CRP good vs. no response group in M-DP scores (log2-transformed) calculated using the analytes in the model
^{c} The mean difference calculated excluding the sirukumab studies SIRROUND- M, -D-, and -T.

TABLE 5

| Pharmacodynamics and response association of the “minimal set” 4-analyte M-DP scores | | | | | | | | |
|---|---|---|---|---|---|---|---|---|
| | | | M-DP score (log2 (fold/baseline): mean ± SD [N] ^{a} | | | | Mean difference ± SD | |
| | M-DP score | | | DAS28-CRP response group^{c} | | | (p-value) ^{d} | Response |
| Study | at week ^{a} | Treatment group^{b} | All treated | Good | Moderate | No | Good vs. No | at week^{c} |
| SIRROUND-M | 4 | Sirukumab 100 | −1.87 ± 0.75 | −1.86 ± 0.69 | −1.99 ± 0.94 | −1.21 ± 0.43 | n/a | 24 |
| | | mg q2w | [61]+ | [44]+ | [15]+ | [2] | | |
| | | Sirukumab 50 | −1.69 ± 0.76 | −1.92 ± 0.63 | −1.34 ± 0.75 | −0.96 ± 0.85 | −0.96 ± 0.66 (0.0010) | 24 |
| | | mg q4w | [61]+ | [41]+ | [13]+ | [7] * | | |
| SIRROUND-D | 4 | Sirukumab 100 | −1.21 ± 0.78 | −1.39 ± 0.75 | −1.01 ± 0.75 | −0.92 ± 0.81 | −0.47 ± 0.76 (0.0041) | 24 |
| | | mg q2w | [201]+ | [116]+ | [61]+ | [28]+ | | |
| | | Sirukumab 50 | −1.09 ± 0.80 | −1.26 ± 0.78 | −1.05 ± 0.76 | −0.79 ± 0.81 | −0.47 ± 0.79 (0.0012) | 24 |
| | | mg q4w | [205]+ | [98]+ | [59]+ | [44] + | | |
| | | Placebo | −0.06 ± 0.42 | −0.07 ± 0.66 | −0.09 ± 0.30 | −0.05 ± 0.33 | −0.02 ± 0.45 (0.8286) | 24 |
| | | | [118] | [27] | [29] | [62] | | |
| | 24 | Sirukumab 100 | −1.79 ± 0.78 | −1.80 ± 0.80 | −1.84 ± 0.95 | −1.59 ± 0.19 | n/a | 24 |
| | | mg q2w | [28]+ | [20]+ | [5]* | [2] | | |
| | | Sirukumab 50 | −1.43 ± 0.82 | −1.50 ± 0.83 | −1.46 ± 0.57 | 0.27 ± 0.00 | n/a | 24 |
| | | mg q4w | [27]+ | [20]+ | [7]* | [1] | | |
| | | Placebo | −0.55 ± 0.96 | −0.18 ± 0.48 | −0.07 ± 0.39 | −1.11 ± 1.19 | n/a | 24 |
| | | | [33] * | [11] | [8] | [14] * | | |
| SIRROUND-T | 4 | Sirukumab 100 | −0.79 ± 0.76 | −1.13 ± 0.80 | −1.05 ± 0.93 | −0.79 ± 0.83 | −0.34 ± 0.81 (0.0416) | 24 |
| | | mg q2w | [134]+ | [65]+ | [30]+ | [39] + | | |
| | | Sirukumab 50 | −1.02 ± 0.84 | −0.91 ± 0.75 | −0.82 ± 0.79 | −0.50 ± 0.70 | −0.39 ± 0.73 (0.0126) | 24 |
| | | mg q4w | [127]+ | [63]+ | [34]+ | [30] * | | |
| | | Placebo | −0.07 ± 0.36 | −0.11 ± 0.33 | −0.40 ± 0.43 | −0.02 ± 0.36 | −0.09 ± 0.35 (0.3521) | 24 |
| | | | [56] | [24] | [3] | [29] | | |
| | 24 | Sirukumab 100 | −1.03 ± 0.83 | −1.18 ± 0.80 | −1.19 ± 0.90 | −0.74 ± 0.75 | −0.44 ± 0.78 (0.0067) | 24 |
| | | mg q2w | [136]+ | [65]+ | [31]+ | [40] + | | |
| | | Sirukumab 50 | −0.85 ± 0.81 | −0.98 ± 0.81 | −0.88 ± 0.81 | −0.55 ± 0.76 | −0.43 ± 0.79 (0.0167) | 24 |
| | | mg q4w | [128]+ | [63]+ | [34]+ | [31] * | | |
| | | Placebo | 0.03 ± 0.51 | −0.16 ± 0.48 | −0.36 ± 0.65 | 0.23 ± 0.45 | −0.39 ± 0.46 (0.0040) | 24 |
| | | | [56] | [24] | [3] | [29] * | | |
| SIRROUND-H | 4 | Sirukumab 50 | −0.88 ± 0.69 | −0.92 ± 0.65 | −0.95 ± 0.74 | −0.52 ± 0.41 | −0.40 ± 0.59 (0.0549) | 24 |
| | | mg q4w | [96]+ | [29]+ | [54]+ | [12]* | | |
| | | Adalimumab 40 | −0.43 ± 0.53 | −0.58 ± 0.50 | −0.39 ± 0.59 | −0.29 ± 0.37 | −0.29 ± 0.45 (0.0217) | 24 |
| | | mg q2w | [100] + | [34]+ | [43] + | [23]* | | |
| GO-FURTHER | 14 | Golimumab IV | −0.94 ± 0.63 | −1.13 ± 0.61 | −0.94 ± 0.68 | −0.57 ± 0.43 | −0.56 ± 0.55 (0.0003) | 24 |
| | | | [100]+ | [40]+ | [38]+ | [22]+ | | |
| CNTO1275ARA2001 | 4 | Ustekinumab | −0.05 ± 0.77 | −0.49 ± 0.27 | 0.06 ± 0.41 | 0.33 ± 0.98 | −0.81 ± 0.83 (0.0469) | 24 |
| | | | [36] | [14]+ | [9] | [31] | | |
| | | Guselkumab | −0.12 ± 0.39 | −0.19 ± 0.30 | −0.11 ± 0.49 | −0.08 ± 0.35 | −0.11 ± 0.33 (0.4781) | 24 |
| | | | [31] | [7] | [12] | [12] | | |
| | | Placebo | −0.06 ± 0.34 | −0.03 ± 0.53 | −0.01 ± 0.20 | −0.09 ± 0.39 | 0.06 ± 0.41 (0.7604) | 24 |
| | | | [50] | [5] | [19] | [26] | | |
| | 28 | Ustekinumab | 0.04 ± 0.84 | −0.53 ± 0.68 | −0.04 ± 0.53 | 0.41 ± 0.94 | −0.94 ± 0.82 (0.0323) | 24 |
| | | | [36] | [14] * | [9] | [13] | | |
| | | Guselkumab | −0.07 ± 0.37 | −0.12 ± 0.38 | −0.10 ± 0.39 | −0.01 ± 0.38 | −0.12 ± 0.38 (0.5645) | 24 |
| | | | [31] | [7] | [12] | [12] | | |
| | | Placebo | −0.18 ± 0.41 | 0.14 ± 0.54 | 0.22 ± 0.30 | −0.21 ± 0.45 | 0.07 ± 0.46 (0.1362) | 24 |
| | | | [50] * | [5] | [19]* | [26] * | | |
| 40346527ARA2001 | 4 | JNJ-40346527 | −0.03 ± 0.49 | −0.29 ± 0.42 | −0.05 ± 0.48 | −0.09 ± 0.54 | 0.34 ± 0.52 (0.1581) | 12 |
| | | | [61] | [5] | [28] | [23] | | |
| | | Placebo | −0.01 ± 0.33 | −0.02 ± 0.55 | −0.07 ± 0.28 | 0.10 ± 0.26 | −0.08 ± 0.39 (0.5683) | 12 |
| | | | [31] | [6] | [14] | [10] | | |
| | 12 | JNJ-40346527 | −0.01 ± 0.56 | −0.50 ± 0.61 | −0.07 ± 0.55 | 0.18 ± 0.50 | −0.68 ± 0.52 (0.0079) | 12 |
| | | | [56] | [6] | [27] | [22] | | |
| | | Placebo | −0.14 ± 0.34 | −0.06 ± 0.49 | −0.18 ± 0.33 | −0.15 ± 0.28 | 0.09 ± 0.37 (0.6311) | 12 |
| | | | [30] * | [6] | [14] | [10] | | |
| TACERA | 26 | csDMARDs | −0.69 ± 0.63 | −0.50 ± 0.36 | −0.79 ± 0.39 | −0.06 ± 0.31 | −0.60 ± 0.35 (0.0003) | 26 |
| | | | [95]+ | [50]+ | [23]+ | [22] | | |

^{a} M-DP scores evaluated at indicated week, reported as log2 (fold/baseline), for 4-analyte M-DP panel of CRP + CXC10 + CXCL13 + SAA.
* p < 0.05 and † p < 0.0001 vs. baseline (paired t-test).
Bolded when absolute value of mean M-DP score >= SD.
^{b} Sirukumab 100 mg q2w, Sirukumab subcutaneous 100 mg q2 weeks; Sirukumab 50 mg q4w, Sirukumab subcutaneous 50 mg q4 weeks; Golimumab IV, Golimumab intravenous 2 mg/kg at Weeks 0 and 4, then q8 weeks thereafter pl us concomitant weekly methotrexate; Ustekinumab, Ustekinumab subcutaneous 90 mg at Weeks 0, 4, then q8 weeks, ustekinumab sc 90 mg at Weeks 0, 4, then every 12 (ql2) weeks; Guselkumab, Guselkumab sc 200 mg at Weeks 0, 4, then q8 weeks, or guselkumab sc 50 mg at Weeks 0, 4, then q8 weeks; JNJ-40346527, JNJ-40346527 oral 100 mg twice daily (BID); esDMARDs, Conventional synthetic DMARDs (no DMARDs prior to baseline visit): hydrochloroquine only (9%), methotrexate ± hydrochloroqine (76%), methotrexate + sulfasalizine/prednisone (8%), sulfasalazine only (4%), prednisone only (3%)
^{c} DAS28-CRP response evaluated at the week indicated in column ″Response at week″
^{d} Difference of group means ± standard deviation (p-value for comparison) between DAS28-CRP EULAR good vs. no response groups for log2 (fold/baseline) values of 4-analyte M-DP scores (CRP ± CXCL1 ± CXC:13 ± SAA panel). Bolded when p < 0.05.

TABLE 6
Pharmacodynamics and response association of U-PLEX 4-analyte M-DP scores for abatacept and rituximab treatments

| | | M-DP score (log2 (fold/baseline): mean ± SD [N] ^{b} | | | | Mean difference ± |
|---|---|---|---|---|---|---|
| | M-DP score | | 6-month DAS28-CRP response group | | | SD (p-value) ^{c} |
| Treatment ^{a} | at month | All treated | Good | Moderate | No | Good vs. No |
| Abatacept | 3M | −0.87 ± 1.01 [38] + | −1.04 ± 1.07 [19] + | −0.96 ± 0.97 [12] * | −0.25 ± 0.71 [7] | −0.79 + 0.99 (0.0044) |
| | 6M | −0.85 ± 1.09 [38] + | −1.21 ± 0.95 [19]+ | −0.84 ± 1.10 [12] * | 0.13 ± 0.94 [7] | −1.34 ± 0.94 (0.0084) |
| Rituximab | 3M | −0.51 ± 1.20 [12] | −0.44 ± 1.13 [7] | −0.71 ± 1.62 [4] | −0.23 ± n.a. [1] | n.a. |
| | 6M | −0.64 ± 0.95 [12] * | −0.96 ± 0.79 [7]* | −0.24 ± 1.23 [4] | −0.01 ± n.a. [1] | n.a. |

^{a} Serum samples from the CORRONA CERTAIN biorepository were analyzed for RA patients at baseline (pre-treatment) and through 3- and 6-months of treatment with abatacepror rituximab
^{a} M-DP scores evaluated at indicated month, reported as log2 (fold/baseline), for 4-analyte M-DP panel of CRP+CXC10+CXCL13+SAA meausred by MSD U-PLEX platform.
* p < 0.05 and † p < 0.0001 vs. baseline (paired t-test).
Bolded when absolute value of mean M-DP score >= SD.
^{c} Difference of group means ± standard devation (p-value for comparison) between 6-month DAS28-CRP EULAR good vs. no response groups for log2 (fold/baseline) values of the M-DP scores. Bolded when p < 0.05.

TABLE 7
Correlation between change in DAS28-CRP and pharmacodynamic M-DP scores

| | | DAS28-CRP | 14-analyte M-DP | | 4-analyte M-DP | |
|---|---|---|---|---|---|---|
| Study | Treatment group | change | Week 4 | Week 12−28 | Week 4 | Week 12−28 |
| SIRROUND-M | Sirikumab 100 mg q2w | Week 24 | 0.33 (0.011)a | n.a. | 0.46 (0.002) | n.a. |
| | Sirikumab 50 mg q4w | Week 24 | 0.30 (0.018) | n.a. | 0.32 (0.013) | n.a. |
| SIRROUND-D | Sirikumab 100 mg q2w | Week 24 | 0.14 (0.049) | 0.19 (0.33) | 0.26 (0.0002) | 0.20 (0.32) |
| | Sirikumab 50 mg q4w | Week 24 | 0.17 (0.017) | 0.34 (0.040) | 0.34 (<0.0001) | 0.45 (0.017) |
| | Placebo | Week 24 | 0.26 (0.0042) | 0.26 (0.013) | 0.13 (0.16) | 0.37 (0.033) |
| SIRROUND-T | Sirikumab 100 mg q2w | Week 24 | 0.30 (0.0005) | 0.34 (<0.0001) | 0.27 (0.0017) | 0.33 (0.0001) |
| | Sirikumab 50 mg q4w | Week 24 | 0.13 (0.16) | 0.31 (0.0004) | 0.29 (0.0009) | 0.35 (<0.0001) |
| | Placebo | Week 24 | 0.20 (0.14) | 0.60 (<0.0001) | 0.24 (0.076) | 0.49 (0.0001) |
| SIRROUND-H | Adalimumab 40 mg q2w | Week 24 | 0.22 (0.027) | n.a. | 0.28 (0.0047) | n.a. |
| | Sirikumab 50 mg q4w | Week 24 | 0.20 (0.048) | n.a. | 0.22 (0.031) | n.a. |
| GO-FURTHER | Golimumab IV | Week 24 | n.a. | 0.35 (0.0003) | n.a. | 0.37 (0.0002) |
| TACERA | csDMARDs | Week 26 | n.a. | 0.47 (<0.0001) | n.a. | 0.45 (<0.0001) |
| CNTO1275ARA2001 | Ustekinumab | Week 24 | 0.45 (0.015) | 0.60 (0.0006) | 0.50 (0.0059) | 0.56 (0.0014) |
| | Guselkumab | Week 24 | 0.11 (0.54) | 0.06 (0.75) | 0.30 (0.10) | 0.19 (0.30) |
| | Placebo | Week 24 | 0.02 (0.88) | 0.26 (0.064) | 0.05 (0.74) | 0.00 (0.99) |
| 40346527ARA2001 | JNJ-40346527 | Week 12 | 0.20 (0.13) | 0.45 (0.0005) | 0.04 (0.78) | 0.47 (0.0002) |
| | Placebo | Week 12 | 0.30 (0.11) | 0.14 (046) | 0.16 (0.40) | 0.24 (0.20) |

^{a} Pearson’s correlation coefficient R (p-value) for correlation between change in DAS28-CRP at indicated week and log2 (fold/baseline) M-DP scores, for the 14- or 4- analyte panels, at week 4 or week 12−28 (week 24 for SIRROUND -M, -D-, T-, and - H; week 14 for GO-FURTHER; week 26 for TACERA; week 28 for CNTO1275ARA2001; week 12 for 40316527ARA2001).
Bolded when R > 0.30 and p < 0.05.

### Sample Size Estimates to Power Decisional M-DP Score Outcomes

Based on the above results, it is hypothesized that an efficacious treatment should significantly decrease in M-DP scores by 4-weeks of treatment. The smallest effect size for an efficacious treatment was for adalimumab at week 4, with an M-DP score equivalent to a decrease of 0.8 standard deviations. Effect sizes for all other efficacious treatments had a decrease of at least 1.0 standard deviations, albeit at week 14 for golimumab IV and week 26 for csDMARDs M-DP scores, with week 4 data not available. EULAR DAS28-CRP good responders to ustekinumab had greater than a 1.0-standard deviation decrease for week 4 M-DP scores, supporting the hypothesis that significant decreases in M-DP scores at week 4 are reflective of efficacious treatments.

To estimate the number of patients needed in a study to prospectively evaluate whether 4-analyte M-DP scores significantly decrease to the extent observed for efficacious treatments, power calculations were performed under various scenarios. With the underlying assumption that the true M-DP effect sizes for efficacious treatments are 0.8 or 1.0 standard deviations in magnitude, then a study with 26 or 17 patients per group, respectively, would have 80% power to detect a difference between an efficacious treatment vs. placebo at a significance level of p=0.05 (Table S3). 20 or 14 patients per group, respectively, would be needed to detect a difference at a significance level of p=0.10 (Table S3).

TABLE S3
Sample size estimates for M-DP scores as study outcomes

| | Effect | vs. 0- | |
|---|---|---|---|
| P-value | size ^{a} | change ^{b} | vs. placebo ^{c} |
| 0.05 | 1.0 SD's | 10 | 17 |
| | 0.8 SD's | 15 | 26 |
| 0.10 | 1.0 SD's | 8 | 14 |
| | 0.8 SD's | 12 | 20 |

^{a} Effect size of 1.0 standard deviations (SD's) were observed for golimumab at week 14 and csDMARDs at week 26, with larger effect sizes for sirukumab (at week 4), whereas effect size of 0.8 SD's was observed for adalimumab at week 4
^{b} Number of patients estimated to be needed for active treatment group to observe at indicated significance level with 80% power a difference for log2(fold/baseline) values with active treatment vs. 0-change from baseline, assuming true difference of indicated effect size (one-sample t-test)
^{c} Number of patients estimated per treatment group (active, placebo) to observe at indicated significance level with 80% power a difference for log2(fold/baseline) values with active treatment vs. placebo treatment assuming true difference of indicated effect size (two-sample t-test)

Because the week 4 PD M-DP scores for placebo treatments were minimal in all studies evaluated, with the largest numeric mean decrease observed less than 5%, it may not be necessary to formally compare week4 PD M-DP scores between active and placebo treatment groups. For one-sample t-tests with the null hypothesis of mean M-DP scores=0 with the underlying assumption that the true M-DP effect sizes for efficacious treatments are 0.8 or 1.0 standard deviations in magnitude, then a study with 15 or 10 patients in the active treatment group, respectively, would have 80% power to detect the treatment effect at a significance level of p=0.05 (Table S3). 12 or 8 patients per group, respectively, would be needed to detect a difference at a significance level of p=0.10 (Table S3).

Patients with non-response to efficacious treatments had significantly lesser mean decreases in M-DP scores compared to good responders. It was therefore hypothesized that if the patients in the non-response group were switched to a treatment regimen that was then efficacious, then the mean M-DP scores for this group should demonstrate a significant further decrease at least to levels observed in good responders to the original treatment. Based on the golimumab treatment data, the good response subgroup had a 54% decrease compared to a 33% decrease for the no response subgroup. In a hypothetical study with patients who had no response to a run-in golimumab treatment and were then switched to a more effective therapy in which all patients in the subgroup then achieved good response, a further 32% mean decrease in M-DP scores would be expected in this subgroup. If only ⅔ or ½ of patients in the subgroup achieved good response after being switched to the new treatment regimen, then a further 23% or 18% mean decrease in M-DP scores, respectively, would be expected. The study would have 80% power to detect significant (p=0.05) further decreases in M-DP scores after switching to an effective treatment regimen with 12, 20, 30 patients in the golimumab run-in non-response subgroup, assuming 100%, 67% or 50% of the patients achieve good response after switching (Table S4). 8, 15, or 23 patients would be needed in the golimumab run-in no response subgroup to detect differences at a significance level of p=0.10 (Table S4).

TABLE S4
Sample size estimates for M-DP scores as combination study outcomes

| | % achieving good response ^{a} | | |
|---|---|---|---|
| p-value | 100% | 67% | 50% |
| 0.05 | 12 ^{b} | 20 | 30 |
| 0.10 | 8 | 15 | 23 |

^{a} Effect sizes for 100%, 67%, and 50% achieving good response would be 0.54, 0.68, and 0.92 standard deviations, respectively (equivalent to 32%, 21%, and 16% decreases from baseline)
^{b} Number of patients estimated to be needed for active treatment group to observe at indicated significance level with 80% power a difference for log2(fold/baseline) values with active treatment vs. 0-change from baseline, assuming true difference of effect size corresponding to the assumption of indicated % achieving good response (one-sample t-test)

### DISCUSSION

### Pharmacodynamics of M-DP Scoring

A set of 14 analytes were identified from SomaLogic SOMAscan® profiling to be significantly and consistently elevated in serum samples of RA patients compared to samples from demographically-matched healthy controls. A composite score for this M-DP (also named PD M-DP) that summarizes the pharmacodynamic effects of treatment on the analyte set, the PD M-DP score, was defined at individual patient-level as the median of the 14 analytes in the M-DP, with each normalized to its value at baseline.

When evaluating the PD M-DP scores across seven interventional clinical trials in established RA patients, it was observed that the M-DP scores were significantly decreased in the active treatment group compared to 0-change from baseline and to the change from baseline in the placebo group (when data from placebo group available), for treatments that were clinically efficacious. These treatments included: sirukumab (4 phase 3 studies, evaluated at week 4 post-baseline), adalimumab (active comparator arm of 1 phase 3 study, evaluated at week 4 post-baseline), and golimumab (1 phase 3 study, evaluated at week 14 post-baseline). Such a significant decrease in M-DP scores was not observed for treatments that were not clinically efficacious, including: ustekinumab and guselkumab (phase 2 study, evaluated at week 4 post-baseline) and CSF1R-antagonist JNJ-40346527 (phase 2 study, evaluated at week 4 post-baseline). A significant decrease in M-DP score was also observed in an early RA cohort after initial 6-month treatment with csDMARDs.

The decision to define the M-DP score based on the median of the normalized values for the set of analytes, rather than modeling coefficients for each analyte was based on the desire to have the M-DP scores be independent of specific treatments. The intention was also to define a score that reflected the molecular burden of disease rather than be a surrogate for clinical activity. For these reasons, M-DP scores were not modeled to optimally reflect the pharmacodynamic effects of a treatment nor associations with clinical response to a treatment, as the model could become too selective for a specific treatment and not more broadly generalizable. Nor was the M-DP score modeled to highly correlate with clinical disease activity, e.g., with DAS28-CRP or CDAI.

A major advantage of the taking the median of normalized values is that no one analyte could be overly influential in the scores. This advantage was confirmed in permutation analyses, in which removal of any one analyte from the 14-analyte M-DP panel did not significantly impact the performance of M-DP scores for pharmacodynamic and clinical response associations. This quality can be important for treatments that can impact a class of analytes much more strongly than other therapeutic classes, e.g., IL-6 inhibitors very strongly decrease CRP to levels even below those observed in a healthy population, whereas TNFi significantly decrease CRP levels but to not such a dramatic extent.

The approach for defining M-DP scoring differed from the methods employed in the development of the Multi-Biomarker Disease Activity (MBDA, marketed as Vectra-DA) test (PMID: 23585841). This test was developed to correlate with clinical disease activity, specifically DAS28-CRP. Of the 12 serum analytes in the MBDA panel, 5 are in common with the currently-described 14-analyte M-DP panel: CRP, IL-6, MMP-1, MMP-3, SAA. Indeed, the M-DP scores were not intended to directly reflect clinical disease activity, with only modest correlation between changes in DAS28-CRP and M-DP scores observed (correlations ranging from r=0.31 to 0.60), compared to r=0.51 in a validation study for the MBDA scores after methotrexate or TNFi treatment (PMID: 22736476). Whether the MBDA test would perform similarly to M-DP scoring has not been established, but given the overlap in analytes, this could indeed be the case although it may be expected that the MBDA score would overestimate effects of treatments that directly impact the acute phase (e.g, IL-6-, TNF-, and IL-1—inhibitors) relative to those that may impact the pathway indirectly. Indeed, although MBDA scores correlated with disease activity after treatment with TNFi in the initial validation studies, in independent evaluations, the MBDA test overestimated improvements in clinical activity for adalimumab compared to underestimation for abatacept, despite both treatments having nearly equivalent impact on DAS28-CRP scores (PMID: 27111089).

### Clinical Response Associations

M-DP scores were decreased significantly more in EULAR DAS28-CRP good response group compared to no response group for the clinically efficacious treatments. Although M-DP scores were not significantly decreased in the active treatment group overall for non-efficacious treatments, M-DP scores were decreased in the subset of DAS28-CRP good responders compared to non-responders on active treatment and compared to the placebo group at weeks 4 and 28 for ustekinumab and week 12 (but not week 4) for CSF1R-antagonist.

However, practical classification power to accurately discriminate clinical responders from non-responders is limited by the substantial overlap in M-DP score distributions between clinical response groups. For example, classification of week 24 DAS28-CRP good vs. non-responders using week 4 PD M-DP scores as the predictor had an area-under-receiver-operating-characteristic-curve (AUC-ROC)=0.62, compared to AUC-ROC=0.83 using baseline and week 4 change in DAS28-CRP score as predictors (data not shown). Adding week 4 PD M-DP scores to baseline and week 4 change in DAS28-CRP for predictors did not improve predictive power, with AUC-ROC unchanged.

It is hypothesized that serum RA M-DP scores would be significantly decreased, at population level, for treatments that are clinically efficacious in the overall study population. M-DP scores would not be significantly decreased for treatments that are not clinically efficacious, although the scores could be decreased in the subset of clinical responders. In this situation of decreases in M-DP scores only in clinical responders for an overall non-efficacious treatment, this would support the possibility that the treatment could be efficacious in a subpopulation of patients but would require an adequately designed study to confirm. Although significant changes have been observed at week 4 visits for efficacious treatments (sirukumab, adalimumab) and for the clinical-responder subgroups for ustekinumab, it cannot be established that for different mechanistic classes that significant changes would be observable this early after baseline. Only baseline and 6-month samples were available for the efficacious csDMARD treatment in early RA, and for CSF1R-antagonist clinical responder subset significant changes were observed at week 12 but not yet at week 4.

From permutation analyses, it was demonstrated that no one single analyte was absolutely required to retain the association with treatment response across the studies. Critically, CRP was not necessary to include in the panel, with significance of changes from baseline improving for some studies. Of practical importance, the 14-analyte M-DP panel was able to be reduced to 4 analytes (CRP, SAA, CXCL10, CXCL13) while retaining similar if not superior performance across the studies evaluated.

Using this reduced 4-analyte panel, it was demonstrated that the concurrence of decreases in M-DP scores with clinical efficacy for abatacept and rituximab, which were not previously evaluated for the panel development or reduction. In addition to validating the performance of the 4-analyte panel, this further supports the generalizability of the concurrence of significant decreases in M-DP scores with clinical efficacy regardless of mechanism of action of the treatment.

### Prospective Application to Clinical Studies

Based on this concurrence of significant decreases in M-DP with clinical efficacy at population level, despite lack of predictive power at individual patient level, a practical application for M-DP scoring for clinical study designs can be envisaged. For early-phase interventional studies in RA, a relatively small number of patients could be evaluated for M-DP scores as early as after 4 weeks of treatment. If a significant change relative to baseline is observed with the treatment, it can be presumed that the treatment would be clinically efficacious if treatment continued for a full study period of 24 weeks. Not only would the amount of time that a patient would need to be treated before decisions to proceed to next study be shortened, e.g., from 12 to 24 weeks down to 4 weeks, but also the number of patients required to be treated would be substantially reduced compared to a standard study design evaluating clinical efficacy per se. A major driver allowing for much smaller studies with M-DP scoring as the outcome is the lack of requirement for statistical comparison to a placebo control group, given the consistent lack of impact of placebo treatment on M-DP scores (<5% GMean decrease across 4 available studies). In a similar fashion, M-DP scoring could be used as a futility outcome for an interim analysis, with the decision to proceed to full enrollment made after the first 8-12 patients on active treatment are treated for at least 4 weeks, with the decision to proceed to full enrollment for a primary clinical efficacy outcome based on whether there is a decrease in M-DP scores at a significance level of p<0.10. M-DP scoring could also be potentially powerful in platform or ‘pick-the-winner’ studies, in which multiple treatments would be evaluated in RA concurrently or sequentially, with treatments that do not significantly decrease M-DP scores deprioritized for further evaluations.

Patients with non-response to efficacious treatments had significantly lesser mean decreases in M-DP scores compared to good responders. It was therefore hypothesized that if the patients in the non-response group were switched to a treatment regimen that was then efficacious, then the mean M-DP scores for this group should demonstrate a significant further decrease at least to levels observed in good responders to the original treatment. A study of a combination of treatments, where a second treatment is added onto a primary treatment, e.g., a TNFi treatment, could leverage M-DP scoring to allow for smaller and faster studies. In patients with inadequate response to TNFi, after a run-in period with the TNFi to allow M-DP scores to stabilize to levels expected in TNFi inadequate responders, the second treatment would be added to the TNFi. M-DP scores would be evaluated just before addition of the second treatment and after at least 4 weeks on the combination treatment. A significant decrease in the M-DP scores would provide compelling biological evidence that the add-on treatment would provide clinical efficacy compared to continuing with the primary treatment per se.

### Concluding Remarks

RA M-DP scoring with a panel of 4 serum analytes can be utilized in small, early phase proof-of-mechanism studies to make relatively quick decisions about potential clinical efficacy or for interim futility analyses in larger phase 2 studies to make decisions about whether to continue to fully enroll the study or terminate the study early. Placebo comparator arms are not critical for the evaluations, allowing for reduced sample sizes. The estimated sample size needed per active treatment arm is 10-15 patients at a significance of p=0.05, or 8-12 patients at a significance of p=0.10. RA M-DP scoring could also be potentially powerful in platform or ‘pick-the-winner’ studies, in which treatments that do not significantly decrease M-DP scores are deprioritized. Application to clinical studies of combination treatment approaches could also leverage the observation of greater decreases in M-DP scores in good clinical responders compared to non-responders.

1. An isolated set of probes for detecting a panel of biomarkers consisting of two, three, four, five, six, seven, eight, nine, ten, eleven, twelve, thirteen or fourteen biomarkers selected from: C-reactive protein pentraxin-related (CRP), serum amyloid A-1 protein (SAA), C-X-C motif chemokine 10 (CXCL10), C-X-C Motif Chemokine Ligand 13 (CXCL13), interleukin 6 (IL-6), phosphohexose isomerase (PHI), annexin I, BPI (bactericidal/permeability-increasing protein), LEAP-1 (liver-expressed antimicrobial protein), MMP-1 (metalloproteinase-1), MMP-3 (metalloproteinase-3), PBEF (pre-B-cell colony-enhancing factor 1), SP-D (surfactant protein D), and TIMP-3 (tissue inhibitor of metalloproteinase 3), preferably, the panel consists of CRP, SAA, CXCL10 and at least one of CXCL13, IL-6 and PHI.

2. The isolated set of probes of claim 1, wherein the panel of biomarkers consists of CRP, SAA, CXCL10 and CXCL13; CRP, SAA, CXCL10 and IL-6; or CRP, SAA, CXCL10 and PHI.

3. The isolated set of probes of claim 1, wherein the probes are selected from the group consisting of an aptamer, an antibody, an affibody, a peptide and a nucleic acid; preferably, the probes are labeled with one or more detectable markers.

4. A kit comprising the isolated set of probes of claim 1.

5. A method comprising:
(a) applying the isolated set of claim 1 to a biological sample to thereby measure quantitative data for each biomarker of the panel of biomarkers in the biological sample, wherein the biological sample is obtained from a subject in need of a treatment of rheumatoid arthritis at a time point T1 after the subject is treated with a therapy;
(b) obtaining a treatment dataset comprising the quantitative data for all biomarkers of the panel of biomarkers measured in (a);
(c) obtaining a baseline dataset comprising quantitative data for all biomarkers of the panel of biomarkers, preferably the baseline dataset comprises quantitative data for all biomarkers of the panel of biomarkers measured from a biological sample obtained from the subject before the subject is treated with the therapy;
(d) comparing the quantitative data in the treatment dataset with the corresponding quantitative data in the baseline dataset to obtain a change in each biomarker of the panel of biomarkers at the time point T1 after the subject is treated with the therapy; and
(e) determining the molecular disease profile (M-DP) score of the subject at the time point T1 as the median value of the changes in all biomarkers of the panel of biomarkers measured in (d).

6. The method of claim 5, wherein the M-DP score is determined as the median value of the log₂ transform of the ratio of the quantitative data for each biomarker in the treatment dataset over the corresponding quantitative data in the baseline dataset.

7. The method of claim 5, further comprising:
(f) determining an M-DP score for the subject at least one additional time point T2 after the subject is treated with the therapy using the method of claim 5;
(g) determining a composite M-DP score for the subject as the mean value of the M-DP scores at the time point T1 and the at least one additional time point T2 after the subject is treated with the therapy; and
(h) predicting the efficacy of the therapy in treating rheumatoid arthritis in the subject based on the composite M-DP score for the subject.

8. The method of claim 5, further comprising:
(f) determining an M-DP score for each subject of a group of subjects in need of a treatment of rheumatoid arthritis at the time point T1 after the group of subjects are treated with the therapy, wherein the M-DP score is determined using the method of claim 5;
(g) determining a composite M-DP score for the group of subjects at the time point T1 as the mean value of the M-DP scores for all subjects in the group of subjects at the time point T1; and
(h) predicting the efficacy of the therapy in treating rheumatoid arthritis based on the composite M-DP score.

9. The method of claim 8, wherein the group of subjects consists of 5 to 25 subjects, preferably 10 to 15 subjects, such as 10, 11, 12, 13, 14 or 15 subjects.

10. The method of claim 5, wherein the panel of biomarkers consists of CRP, SAA, CXCL10 and CXCL13.

11. The method of claim 5, wherein the biological sample is a serum sample.

12. The method of claim 7, wherein the composite M-DP score is correlated with a clinical assessment; preferably, the clinical assessment is selected from the group consisting of a DAS, a DAS28, a DAS28-CRP, a Sharp score, a tender joint count (TJC), a swollen joint count (SJC), a Clinical Disease Activity Index (CDAI), and a Simple Disease Activity Index (SDAI); more preferably the clinical assessment is the DAS28-CRP.

13. The method of claim 5, wherein the time point T1 is about 4 to 12 weeks after the subject is treated with the therapy; preferably the time point T1 is about 4 to 8 weeks, such as 4, 5, 6, 7, or 8 weeks, or anytime in between, alter the subject is treated with the therapy.

14. The method of claim 7, wherein the efficacy of the therapy in treating rheumatoid arthritis is predicted based on the composite M-DP score before the efficacy is detected by a clinical assessment.

15. The method of claim 7, further comprising treating the subject(s) with the therapy, if the therapy is predicted to be effective.

16. The method of claim 7, further comprising treating the subject(s) with another therapy, if the therapy is predicted to be ineffective.

17. A method of treating rheumatoid arthritis in a group of subjects in need thereof comprising:
(a) obtaining a baseline biological sample from each subject in the group of subjects;
(b) applying the isolated set of claim 1 to each of the baseline biological samples to detect a baseline expression level for each biomarker detected by the isolated set of probes;
(c) treating the subjects with a therapy for rheumatoid arthritis;
(d) obtaining a biological sample from each subject in the group of subjects at a time point T1;
(e) applying the isolated set of probes from (b) to each of the biological samples to detect an expression level for each biomarker at time point T1;
(f) determining a molecular disease profile (M-DP) score for each subject, wherein the M-DP score is the median value of the log₂ transform of the ratio of the expression level for each biomarker at time point T1 over the corresponding baseline expression level;
(g) determining a composite M-DP score for the group of subjects as the mean value of the M-DP scores for the group of subjects;
(h) continuing to treat the subjects with the therapy in (c) if the composite M-DP score is greater than one standard deviation below zero; or treating the subjects with a different therapy if the composite M-DP score is less than one standard deviation below 0, unchanged, or above zero.

18. The method of claim 17, wherein the time point T1 is about 4 to about 12 weeks after the baseline time; preferably wherein time point T1 is about 4 to about 8 weeks, such as 4 weeks, 5 weeks, 6 weeks, 7 weeks, 8 weeks, or any time point in between, after the baseline time.

19. The method of claim 17, wherein the biological sample is a serum sample.

20. The method of claim 17, wherein the group of subject consists of 5 to 25 subjects, preferably 10 to 15 subjects, such as 10, 11, 12, 13, 14 or 15 subjects.
